(19) United States
(12) Patent Application Publication
(10) Pub. No.: US 2026/0325090 A1
(43) Pub. Date: Oct. 22, 2026

(54) SYSTEM AND METHOD FOR CORONARY VASCULAR DIAGNOSIS AND PROGNOSIS WITH A BIOMECHANICAL STRESS PROFILING INDEX

(71) Applicant: CORCILLUM PTY LTD, Adelaide (AU)

(72) Inventors: Harry CARPENTER, Adelaide (AU); Hugh CARPENTER, Adelaide (AU)

(73) Assignee: CORCILLUM PTY LTD, Adelaide (AU)

(21) Appl. No.: 19/113,266

(22) PCT Filed: Sep. 21, 2023
(86) PCT No.: PCT/AU2023/050915

§ 371 Date: Mar. 19, 2025

(30) Foreign Application Priority Data
Sep. 29, 2022 (AU) 2022902813

(51) Int. Cl. A61B 6/50; A61B 5/00; A61B 6/00; G06T 7/00; G06T 7/11; G06T 17/20; G16H 15/00; G16H 50/30; G16H 50/50

(52) U.S. Cl. CPC A61B 6/504; A61B 5/0066; A61B 5/0084; A61B 6/5217; A61B 6/5247; A61B 6/5294; G06T 7/0012; G06T 7/11; G06T 17/20; G16H 15/00; G16H 50/30; G16H 50/50; G06T 2207/10024; G06T 2207/20081; G06T 2207/20092; G06T 2207/30048; G06T 2207/30101; G06T 2210/41

(57) ABSTRACT

There is proposed a computer implemented method utilises medical imaging (i.e. invasive coronary angiography, invasive optical coherence tomography or other catheter derived imaging and/or non-invasive computed tomography), to generate a 3D computer model of the artery/vasculature to present a risk score to predict future changes in coronary disease based on artificial intelligence and biomechanical simulations. The computer implemented method preferably analyses images from one or more imaging systems and extracts data on the artery structure and function, including augmenting or simulating missing information, to produce the 3D geometry and predictive result of the artery or artery system.

### FIELD OF THE INVENTION

The present invention relates to a system and method for producing a predictive model of the artery/vasculature of a patient to present a risk analysis to predict future changes in coronary disease and suggests optimal treatment pathways based on artificial intelligence and biomechanical simulations.

### BACKGROUND OF THE INVENTION

Cardiovascular disease is the leading cause of death globally, accounting for around 30% of deaths during 2019. There are often no symptoms of the underlying disease of a person's blood vessels, until they experience a heart attack or stroke.

Furthermore, it is difficult to determine which coronary disease or plaques will progress to a critical state, even when medical imaging is used. Accordingly, this can lead to patients having recurrent heart attacks; roughly one in five people have another heart attack within five years. This therefore increasing hospital admissions and healthcare costs, resulting in an economic impact on society. It is estimated that the cost of cardiovascular disease is around US$1.1 trillion annually across the US, EU and UK and is predicted to increase in the coming years.

There are a number of systems disclosed in the published prior art that have been developed to model coronary physiology and predict coronary disease and plaque build-up in a patient's circulatory system.

The published prior art includes, US20210153945 (CHOI et al) entitled Systems and methods for predicting coronary plaque vulnerability from patient specific anatomic image data. The system of CHOI et al, relates generally to a method of reporting coronary plaque vulnerability from patient-specific anatomic image data. The disclosed method includes the steps of: acquiring anatomical image data of the patient's vascular system; determining hemodynamic feature and biochemical features; and predicting a plaque vulnerability present in the patient's vascular system based on the one or more determined feature vectors.

Other prior art publication that the Applicant is aware of include U.S. Pat. No. 6,047,080 entitled Method and apparatus for three-dimensional reconstruction of coronary vessels from angiographic images, US20210228094 entitled Systems and methods for vascular diagnosis using blood flow magnitude and/or direction, and US20210161384 entitled System and methods for estimation of blood flow characteristics using reduced order model and machine learning.

There are also systems in current use within the field that have predictive capabilities, such as the system used by HeartFlow™ comprising AI applied to CT coronary angiogram imaging (CTCA) to determine pressure drop, including the introducing of fluid dynamics into the calculations.

Another system currently used within the field is produced by Artrya™ and relates to the used of Al for detecting ‘vulnerable’ plaques. The system also uses non-invasive computed tomography coronary angiography (CTCA) imaging.

It is appreciated by relevant experts in the field that non-invasive CTCA imaging systems can be used as a tool to identify patients who have severe artery disease and require an invasive procedure. However, this imaging has no predictive capability and provides no understanding of physiology which led to the aforementioned prior art.

There are also several systems that provide continuous data to a medical practitioner during an invasive procedure such as an invasive angiogram. One such system uses fractional flow reserve (FFR) pressure wires to analyse pressure drop across a plaque. Generally, if the pressure drop is below 0.8, doctors intervene (i.e. use of a stent). A FFR system is used for the PressureWire™ X Guidewire sold in the USA by Abbott Laboratories.

There are also systems to determine pressure drop without the need for an extra invasive wire. One such system has been developed by Medis™, and relates to a virtual quantitative flow ratio (QFR) (i.e. using only angiogram images, thereby preventing the need for a wire). The system disclosed by Medis™ however cannot identify the composition of plaque (i.e. if a narrowing is caused by a fatty or calcified plaque) and has no predictive capability.

It is appreciated by those skilled in the art that assessing anatomy or physiology from angiogram images is non-trivial. Acquiring good quality views is difficult, increases radiation exposure for the patient, is highly susceptible to artefacts, requires manual processes which cannot provide information on plaque composition and provides no predictive capability. Moreover, significantly unwell patients such as those with renal failure cannot be exposed to such radiation doses, limiting the usefulness of approaches which require several views and extended procedure time.

There are invasive imaging systems to visualise cross-sectional anatomy and plaque composition. One such system is optical coherence tomography (OCT). An OCT system is used in the Dragonfly OPTIS™ imaging catheter sold by Abbott Laboratories. Such systems also have limitations, including being unable to see behind fatty plaques due to limited tissue penetration of the light-based imaging (signal attenuation). These systems also have no predictive capacity and face the same limitation as previously mentioned invasive wires.

The published prior art discloses various systems that have been developed assessing the individual biomechanical parameters that relate to the diagnosis of dynamic cardiac angiographic images (4D) and modelling vascularity and stress. These published documents include the paper by Wu, X. et al. “Angiography-Based 4-Dimensional Superficial Wall Strain and Stress: A New Diagnostic Tool in the Catheterization Laboratory,” Frontiers in Cardiovascular Medicine, 2021, Vol. 8, Article 667310, pages 1-13. The study suggested that angiography-based superficial wall dynamics have the potential to identify coronary segments at high-risk of plaque rupture and fracture sites of implanted stents. The friction stress caused by flowing blood acting on the wall is calculated on a static model of coronary arteries, with future proposed developments involving the integration of fast computational techniques to allow online availability of superficial wall strain and stress in the catheterization laboratory.

Another prior art document US 2019/0192012 A1 (Cathworks Ltd) discloses an automated determination of parameters based on vascular images, used to calculate a vascular disease score. The score calculator is configured to, for each potential lesion, determine a vascular state scoring tool (“VSST”) score based on at least one of a size of the potential lesion, a distance of the potential lesion from a branch point in the plurality of vascular segments, and a distance of the potential lesion to an adjacent potential lesion. The example device also includes a user interface configured to display the VSST scores for the potential lesions.

Another U.S. Patent Application to Cathworks Ltd (US 2020/0126229) discloses the use of a vascular score as well as a generating a simulation and a virtual stent. The use of machine learning is suggested in numerous prior art documents, including WO 2021/257906 (Univ Northwestern et al.) and WO 2020/146905 (Lightlab Imaging Inc).

It should be appreciated that any discussion of the prior art throughout the specification is included solely for the purpose of providing a context for the present invention and should in no way be considered as an admission that such prior art was widely known or formed part of the common general knowledge in the field as it existed before the priority date of the application.

### SUMMARY OF THE INVENTION

It is an object of the present invention to provide a method of producing an advanced visualisation and predictive model of at least a part of the artery/vasculature system of a patient to present a risk score to predict future changes in coronary disease based on artificial intelligence and biomechanical simulations. Other objects of the present invention are to overcome at least some of the aforementioned problems, or at least provide the public with a useful alternative. The foregoing objects should not necessarily be considered as cumulative and various aspects of the invention may fulfil one or more of the above objects.

The invention could broadly be understood to comprise a computer implemented method and a computer system on which the method is implemented.

The computer implemented method utilises medical imaging (i.e. invasive coronary angiography, invasive optical coherence tomography or other catheter derived imaging and/or non-invasive computed tomography), to generate a 3D computer model of the artery/vasculature to present a risk score to predict future changes in coronary disease based on artificial intelligence and biomechanical simulations.

The computer implemented method preferably analyses images from one or more imaging systems and extracts data on but not limited to the artery centreline, lumen wall, plaque (fatty lipids or calcification) layers of the artery wall and branch regions to produce a 3D geometry of the artery or artery system.

The computer implemented method preferably analyses images and measured data to extract inputs including but not limited to patient history, medication use, clinical presentation, biochemical signatures and determines physiology including but not limited to blood flow velocity, blood pressure, heart rate, dynamic motion of the arteries and microvessel resistance.

The computer implemented method may preferably take manual user inputs from experienced technicians or clinicians.

The computer implemented method preferably applies these physiological inputs and 3D geometry to carry out one or several artificial intelligence and/or biomechanical simulations in real-time to suggest likely outcomes for a plaque and/or artery and/or patient and whether a patient requires or would benefit from a detailed simulation assessment.

The aforementioned methods shall be considered as ‘level one’ analyses and are displayed through a user interface for interactive visualisation in real-time so that the user is able to better assess a patient's condition.

Upon suggestion by the ‘level one’ analyses and/or request from clinicians the computer implemented method preferably carries out a detailed artificial intelligence embedded biomechanical simulation, analysing up to 69 personalised markers.

The computer implemented method then selectively combines and/or omits metrics in combination with ‘level one’ data through a machine learning decision making process to provide a continuous, multi-dimensional biomechanical stress profiling index (BSPI) throughout plaque/plaques, and/or artery/arteries and/or overall for a patient.

It should be appreciated that the BSPI is continuous and multidimensional in such fashion as to suggest the likelihood of several changes, which do not always require every marker, and does not just provide a number (such as pressure drop) or only suggest an overall endpoint.

The computer implemented method preferably presents the BSPI in several formats including but not limited to a written report, data spreadsheet or interactive visualisation with direct comparisons to similar demographics present in the computer system database.

The aforementioned methods shall be considered a ‘level two’ analyses and are displayed through a user interface for interactive visualisation in real-time so that the user is able to better assess a patient's condition.

The computer implemented method preferably allows personalised markers to also be individually interrogated through the interactive visualisation by the user.

The computer implemented method preferably uses a machine learning process to suggest likely or unlikely treatment pathways based on the BSPI including but not limited to: using balloon angioplasty to restore blood flow and visualising optimal locations for the procedure; inserting a stent to hold an artery open and visualising optimal locations for the procedure; suggesting stent patency or malapposition requiring adjustment and visualising the location; performing coronary artery bypass grafting (CABG); using aggressive medical therapies; modifying lifestyle.

The computer implemented method may preferably present data outputs specific to the type of imaging system used.

The computer implemented method preferably integrates imaging data from different imaging systems, if available, into a single augmented user interface.

In one aspect of the invention, but not necessarily the broadest or only aspect, there is proposed a computer implemented method of producing an advanced visualisation and predictive model to provide a personalised biomechanical stress profiling index for a patient, including the steps of:
a. acquiring images, data and characteristics relating to the patient;
b. constructing a vasculature model of at least some of the patient's arteries;
C. extracting or calculating physiological information from acquired images, data and characteristics relating to the patient;
d. undertaking a lightweight ‘level one’ artificial intelligence and/or biomechanical assessment using the acquired data;
e. using the ‘level one’ results to suggest an optimal pathway or the need for a ‘level two’ analysis;
f. generating an augmented visual display and/or report of the ‘level one’ results to assist the clinician in decision making;
g. undertaking a ‘level two’ artificial intelligence and/or biomechanical simulation for a comprehensive patient assessment using several metrics;
h. using the metrics, acquired imaging, data and characteristics relating to the patient to produce a continuous, multi-dimensional biomechanical stress profiling index of one or several plaques and/or arteries.
i. retraining and updating the ‘level one’ analyses with results from biomechanical stress profiling index;
j. utilising the biomechanical stress profiling index to highlight regions of vulnerable plaque, plaque composition, risk of future growth and destabilisation, and/or vessel changes over time; and
k. generating an augmented visual display and/or report of the index to assist the clinician in decision making and determining patient treatments.

In one form step ‘a.’ may include acquiring imaging information from one or more invasive catheter-based imaging systems such as coronary optical coherence tomography.

In another form step ‘a.’ may include acquiring imaging and gantry orientation and gating information from one or more planes in invasive coronary angiography and/or ventriculography.

In another form step ‘a.’ may include acquiring imaging information from non-invasive computed tomography imaging.

In another form step ‘a.’ may include acquiring continuous measurements such as heartrate, blood pressure and electrocardiogramcluding relevant data from wearable technologies and patient characteristics.

In yet another form step ‘a.’ may include acquiring manual inputs from experienced technicians or clinicians.

In one form step ‘b.’ may also include the steps of automatically:
i. pre-processing a two-dimensional intravascular imaging stack on a computer medium such as a central processing unit or graphical processing unit (CPU or GPU);
ii. scaling and axially stacking the pre-processed and segmented image data into slices in three-dimensions;
iii. communicating the pre-processed and segmented data from the local system over a secure network to a centralised cloud compute or containerised instance;
iv. segmenting the pre-processed image stack on a CPU or GPU using machine learning such as a temporal or three-dimensional neural network to identify vascular structure;
v. classifying and segmenting vascular scaffold(s), if present, in two-dimensional frames and generating a three-dimensional map of the scaffold on a GPU with a generative machine learning model and knowledge of the scaffold design pre-insertion;
vi. implementing a deep physics-informed neural network on a GPU with knowledge of tissue continuity, vascular structure, blood pressure and image properties to reconstruct medial and adventitial layers in attenuated regions;
vii. classifying and segmenting plaque components using a three-dimensional neural network machine learning algorithm;
viii. interpolating and voxelising the segmented data slices;
ix. in another form step ‘vii.’ may include feeding segmented slice data into a neural field to produce a differentiable density map in three-dimensions.
x. generating adaptable mesh from the voxelised/density structure suitable for three-dimensional user interaction and simulation processes; and
xi. communicating the processed steps over a secure network back to the local system.

In another form step ‘b.’ may also include the steps of automatically:
i. pre-processing one or more temporal angiogram and/or ventriculogram acquisitions or image sequences on a CPU or GPU;
ii. communicating the pre-processed and segmented data from the local system over a secure network to a centralised cloud compute or containerised instance;
iii. segmenting epicardial vascular structures using numerical and/or machine learning based algorithms;
iv. inputting the pre-processed image sequence(s) and segmented vascular structure(s) and gantry orientation(s) metadata into an angiographic neural radiance field (ANeRF);
v. generating on a GPU, using the angiographic neural radiance field, a three-dimensional density map of vascular and/or ventricular structures;
vi. creating an adaptable mesh from the three-dimensional density map suitable for three-dimensional user interaction and simulation processes; and
vii. communicating the processed steps over a secure network back to the local system.

In yet another form step ‘b.’ may also include the steps of automatically:
i. pre-processing a stack or stacks of computed tomography images and/or axial, coronal and sagittal planes and associated metadata such as bolus time on a CPU or GPU;
ii. communicating the pre-processed and segmented data from the local system over a secure network to a centralised cloud compute or containerised instance;
iii. segmenting vascular and ventricular structures using numerical and/or machine learning based algorithms;
iv. identifying and segmenting vascular scaffolds using numerical and/or machine learning based algorithms;
v. identifying and segmenting plaque components using numerical and/or machine learning based algorithms;
vi. interpolating and voxelising the segmented data stack;
vii. creating an adaptable mesh suitable for three-dimensional user interaction and simulation processes; and
viii. communicating the processed steps over a secure network back to the local system.

In one form step ‘c.’ may also include the steps of:
i. acquiring and processing a temporal range of images rather than a singular image frame;
ii. analysing acquired or processed temporal image data using probabilistic programming and/or machine learning based algorithms; and
iii. extracting relevant image features as a five-dimensional feature set.

In another form step ‘c.’ may also include the steps of:
i. acquiring and processing a temporal range of patient data or characteristics rather that static data points;
ii. analysing acquired or processed temporal data using probabilistic programming and/or machine learning based algorithms; and
iii. extracting relevant data features as a multi-dimensional feature set.

In one form step ‘d.’ may also include the steps of automatically:
i. collating acquired or extracted data into a feature set or sets;
ii. generating an augmented set of boundary conditions to simulate patient cardiac or vascular load;
iii. analysing the feature set and augmented boundary conditions using physics informed machine learning models to acquire a lightweight subset of estimated biomechanical metrics in real-time;
iv. analysing the feature set(s) using computational statistics and generative machine learning models;
v. visualising the acquired feature set(s), computational statistical models and approach for each data point; and
vi. producing a report or dataset for storage in a local or cloud based electronic medium.

In one form step ‘e.’ may also include the steps of:
i. analysing the feature set(s) from step ‘d.’ using computational statistics, probabilistic programming and/or generative machine learning models;
ii. presenting the feature set(s) and the underlying computational model(s) to the user;
iii. taking manual user inputs from experienced clinicians/technicians including but not limited to selecting or adding appropriate data and computational models suited to the patient;
iv. forecasting a generalised risk profile for the patient;
v. generating a probabilistic scenario for various treatment option(s) and presenting the scenario(s) in a graded fashion from strongest to weakest option;
vi. using the generalised risk profile and probabilistic scenario(s) to recommend or not recommend the use of a detailed ‘level two’ simulation; and
vii. producing a report or dataset for storage in a local or cloud based electronic medium.

In one form step ‘f.’ may also include the steps of:
i. accessing the report and/or dataset in preceding steps from the electronic medium;
ii. loading the user profile or taking manual inputs and formatting the visual display to suit their preset settings;
iii. populating the visual display with the report and/or dataset(s) from steps ‘a.’ to ‘e.’;
iv. automatically highlighting or presenting in a visually appreciable manner the statistically significant or important probabilistic data points;
v. augmenting the display with five-dimensional (three-dimensional space, time, and other metrics) data from one or more acquired datasets;
vi. using colour, shape markers or other visually appreciable methods to interactively highlight important regions throughout the vasculature to the user; and
vii. taking user interaction to alter or enhance the display including opening or closing additional data displays or adding/removing datapoints from the five-dimensional display;

In one form step ‘g.’ may also include the steps of automatically:
i. taking a user command to proceed to a ‘level two’ simulation process;
ii. packaging all data from steps ‘a.’ to ‘f.’ and communicating the packaged data over a secure network to a centralised cloud compute or containerised instance;
iii. generating a coarse and a fine mesh of the vascular structure including but not limited to the lumen, plaque components, vascular wall and epicardial structures;
iv. Defining patient-specific boundary conditions to the mesh structure including but not limited to blood properties and profiles, displacement profiles and electrophysiological profiles; and
v. undertaking a simulation at one time-point and/or one heartbeat and or several heartbeats using acquired and calculated patient data, using continuum mechanics principles such as a fluid-structure interaction technique, a fluid-structure-electrophysical interaction technique, a computational fluid dynamics technique and a solid mechanics technique to determine engineering-based stress measures in the vasculature.

In one form step ‘h.’ may also include the steps of:
i. constructing a feature set from the ‘level two’ engineering-based stress measures;
ii. applying probabilistic programming and machine learning based decision approaches to the ‘level one’ and ‘level two’ feature sets;
iii. calculating using step ‘ii.’ a continuous and multi-dimensional biomechanical stress profiling index on the coarse mesh from step ‘g. iii.’;
iv. extracting from step ‘ili.’ using generative methods a feature set of likely outcomes on the patient, vessel, and plaque level(s) at varying time intervals;
v. adding the ‘level one’ and ‘level two’ feature set(s) to a secure cloud based electronic storage medium; and
vi. communicating the processed steps over a secure network back to the local system.

In one form step ‘i.’ may also include the steps of:
i. retrieving the ‘level one’ and ‘level two’ feature set(s) from the secure cloud based electronic storage medium;
ii. calculating via the centralised cloud compute or containerised instance the variance and/or error between the ‘level two’ and ‘level one’ feature set(s);
iii. taking manual inputs from experienced technicians if variance/error exceeds a set threshold;
iv. retrieving feature sets from the secure cloud based electronic storage medium for all relevant patients;
v. retraining the machine learning based approaches from steps ‘a.’, ‘b.’, ‘c.’, ‘d.’, and the ‘level one’ analysis with the retrieved data from steps ‘i.’ and ‘iv.’;
vi. preferably retraining models from step ‘b.’ with a cross-imaging modality data augmentation approach;
vii. pushing the retrained hyperparameters and/or new machine learning models to the cloud-based machine learning operations (MLOps) pipeline; and
viii. communicating updated parameters via an electronic network to the local systems.

In one form step ‘k.’ may also include the steps of:
i. Taking manual inputs to adapt the visualisation to each users preferences;
ii. Visualising the two-dimensional image(s) stacks from one or several imaging modalities;
iii. Visualizing the three-dimensional vasculature from one or several imaging modalities;
iv. Identifying with shape or colour or other visually appreciable markers regions of interest or data points for the user;
v. Taking manual user interactions with markers to display additional information such as predictive graphs or datapoints;
vi. Automatically selecting and displaying the most important data to the user by using the most critical pieces of information designated or extracted from the decision-making process in previous embodiments rather that a static data point.
vii. Presenting data or metrics in both three-dimensions or modified two-dimensional visualisations such as in ‘unwrapped’ views; and
viii. Allowing interactive inputs to move or rotate or zoom two and three-dimensional visualisation of the vasculature in space and time.

In another aspect of the invention there is proposed a computer implemented method of automating the processing and extraction of key features from intravascular imaging including overcoming significant imaging system limitations which include the steps of:
a. Acquiring an intravascular imaging pullback/image stack and associated data at the time of acquisition including but not limited to blood pressure, heart rate and imaging system physics based partial differential equations;
b. Pre-processing the image stack to remove unwanted regions and preferably prefilter noise or artefacts;
c. Passing the pre-processed imaging stack and acquired data/physics knowledge to general computing hardware such as a suitable graphical processing unit;
d. Segmenting the lumen using a spatio-temporal U-Net machine learning architecture that that leverages long-short term memory (LSTM) and attention mechanisms for robustness and generalisation strength in sparse and noisy real-world data;
e. In another embodiment the machine learning model also applies dynamic vertical layering to modify the model layers during processing to improve segmentation;
f. In yet a further embodiment adaptive block contraction is applied to further reshape encoder or decoder architecture during processing to improve segmentation;
g. Masking the preprocessed image stack with the resulting lumen segmentation map;
h. Feeding the masked and preprocessed image stack to a three-dimensional U-Net based machine learning architecture to segment the medial layer of the vessel wall;
i. Passing the preprocessed image stack, lumen segmentation map and medial layer segmentation map to a pre-processing module of a modified deep physics informed neural network architecture;
j. Processing the three-dimensional pixel location, pixel multilayer colour data and multiple segmentation maps through stage one multilayer perceptron's;
k. Extracting global properties from the multilayer perceptron's and acquiring further data from the pre-processing module including a smoothed three-dimensional vessel centerline;
l. Passing local spatial information to the stage two multilayer perceptron's which have access to partial differential equations governing tissue continuity, nonlinear tissue properties, imaging physics-based properties and pressure distributions;
m. Imposing boundary or initial conditions on the network to aid convergence such as by using segmentation masks from step ‘d.’ and ‘h.’;
n. Minimising the network loss function to extract segmented tissue maps including in areas with significant imaging attenuation artifacts and information on tissue properties; and
o. Performing the above steps within a single operation on a graphical processing unit for rapid and efficient processing.

In another aspect of the invention there exists a computer implemented method of automatically generating a three-dimensional density map or anatomical model of the vasculature from invasive coronary angiography (with as little as a single view) via an angiographic neural radiance field (ANeRF) to minimise patient radiation exposure while amplifying available information to the clinician including the steps of:
a. Acquiring at least one invasive angiographic view of the vasculature containing one or several images over the cardiac cycle;
b. Extracting C-arm orientation metadata from the acquired image data including but not limited to primary and secondary angles, detector properties, x-ray properties and source location with respect to the patient/gantry icocenter and the detector plane;
c. Preprocessing the angiographic image or image stack with a machine learning model or numerical method to identify vascular structures;
d. Developing a multigrid or sub-pixel representation of the vascular structures identified in step ‘c.’ to improve render resolution;
e. In another embodiment if multiple acquisitions from varying primary or secondary angles are present then steps ‘a.’ through ‘d.’ will be carried out on each acquisition;
f. In another embodiment multiple views are aligned using their C-arm coordinate metadata extracted in step ‘b.’;
g. In yet another embodiment these views are aligned using an energy minimisation algorithm to overcome acquisition setting errors, c-arm gantry motion, patient motion including from breathing/heart movement and from table or detector panning;
h. Providing the multiscale representation of the angiographic image(s), binary masks and associated C-arm gantry orientation (after alignment with the energy minimisation algorithm) as inputs to the angiographic neural radiance field;
i. Rendering in three-dimensions the density field of the vasculature;
j. In one embodiment the three-dimensional density field is generated with inclusion of three-dimensional vascular connectedness filters to enhance vascular structures and reduce noise;
k. In another embodiment the density field may be processed into voxelised or mesh-based visualisation techniques; and
l. Interactively visualising the three-dimensional anatomy.

In another aspect of the invention there exists a computer implemented method of acquiring transient information from invasive coronary angiography imaging and the previously illustrated embodiments to determine virtual microvessel function, virtual vessel strain, virtual ejection fraction and other functional metrics without the need for further tests or invasive wires including the steps of:
a. Developing the three-dimensional density field of the vasculature using the immediately preceding aspect of the invention for automatically generating a three-dimensional density map or anatomical model of the vasculature from invasive coronary angiography;
b. Identifying background features across angiographic frames including but not limited to ribs or spinal bones;
c. Applying rigid body transformations to co-register background features across image frames to account for C-arm gantry or patient motion;
d. In one embodiment co-registration may produce an augmented set of images representing a two-dimensional space larger than any individual image frame;
e. In another embodiment the co-registration may produce a variable set of C-arm gantry orientations to account for motion artefacts across several image frames;
f. Mapping forward and backward facing images from one or several angiographic frames to the static three-dimensional density field;
g. In another embodiment the co-registered image stack may be used to generate a unique three-dimensional density field for each set of frames over time;
h. In yet another embodiment the static density field may preferably be encoded with continuity constraints and deformed over time to mimic the two-dimensional co-registered image stack;
i. Fitting a predefined myocardial map to the three-dimensional density field;
j. Deforming the fitted myocardial map over one or several cardiac cycles to estimate ventricular function such as ejection fraction;
k. In one embodiment a ventriculogram may be available and may be used to optimise the predefined myocardial map or ventricular estimates;
l. Reprocessing the density field to extract volumetric changes in the density of vascular structures over time;
m. In another embodiment the angiographic neural radiance field (ANeRF) may preferably be modified with an additional multilayer perceptron and Navier-Stokes and continuity-based loss function(s) to encode blood dynamics to the vascular density field;
n. Calculating the dissipation or change in density of the vascular density field;
o. Mapping the dissipation or density changes to specific vessels or vessel segments or myocardial segments;
p. In another embodiment nonvascular regions may be interrogated for changes in density in two or three-dimensions; and
q. In such an embodiment the identified dissipation or density changes may be graded and mapped to vascular structures or myocardial segments as areas of ‘blush’ or microvessel dysfunction.

In another aspect of the invention there is a computer implemented method of providing novel intraluminal or intrastructural biomechanical based metrics that are tailored to specific patients but can be generalised and compared directly between various patients including the steps of:
a. Generating an augmented set of boundary conditions based on patient characteristics;
b. Carrying out a biomechanical simulation or machine learning implemented method to determine the continuum mechanics-based tensor field in fluid or structural domains using the augmented boundary conditions;
c. Calculating isosurfaces of normalised metrics of interest which may include traditional or novel metrics from several equally spaced units within the domain −1 to 1 or 0 to 1;
d. Taking one or several plane-based slices of one or all isosurfaces from step ‘c.’ and determining the area contained within each plane based isosurface slice;
e. In another embodiment of ‘d.’ takes one or several plane based slices of one or all isosurfaces from step ‘c.’ and determining the area contained within the positive and negative isosurface plane based regions;
f. Determining the cross-sectional area of the vessel at one or several planes used in steps ‘d.’ and ‘e.’;
g. Calculating the ratio of isosurface plane/slice based area to the lumen area or the ratio of area of the positive isosurface slice to the area of the negative isosurface slice from one or several domain units;
h. In another embodiment calculating the augmentation variability of the ratio of isosurface and/or lumen plane area across one or several domain units across the range of augmented boundary conditions imposed from step ‘a.’; and
i. Generating a visual display or graph or report of the augmented intraluminal biomechanical based metrics.

In yet another aspect of the invention there exists a computer implemented method of selecting, distributing and using available data to predict or identify outcomes or features in a patient's vasculature including the steps of:
a. Acquiring various input metrics identified throughout illustrated and enclosed embodiments;
b. Determining the statistical or probabilistic spatio-temporal distributions of continuous metrics;
c. Multi-level discretisation of the statistical or probabilistic spatio-temporal distributions to highlight or improve weighting on important locations or results that may otherwise be overlooked or outweighed;
d. Binning discretised or whole metrics in a multi-level, multi-variable feature binning process;
e. Weighting or shifting bins using patient characteristics for optimal capture of data from one or several metrics;
f. Implementing the bins as inputs or hidden layers in a fully connected network to capture nonlinear features and interactions;
g. Automatically prune connections to optimise the propagation of features through the network preferably in parallel but also in serial processes; and
h. Provide the likelihood of an outcome, the location or statistical probability of a certain feature being present or the probability or predicted success rate of one or several intervention(s) or treatment pathway(s) for multiple parallel endpoints.

The visual display may be generated by a designated visualisation tool or designated hardware.

Preferably, geometrical/morphological based metrics relating to one or several vessels or plaques may be selected for visualisation or further analytics from a group including, but not limited to: Volume; Torsion; Curvature; Stenosis percentage; Minimum lumen area; Lesion diffusivity; Lesion length; Branch angulation; Ostium position; Plaque composition (lipidic, calcific, fibrotic, necrotic, complex); Epicardial adipose tissue; Plaque eccentricity; Lipid volume; Lipid length; Calcium volume; Fibrous cap thickness; Cholesterol crystal presence; Microchannel presence; Macrophage index; Thrombus presence; Rupture presence; Vessel wall thickness (intima, media, adventitia); and subsequent derivations from these metrics such as percent atheroma volume and as outlined in the illustrated embodiments.

The geometrical/morphological based metrics may further be selected from a group including the transient variation of each metric over one or several partial or full cardiac cycles.

The functional based metrics may be calculated from angiogram images and measured ECG and blood pressure data and may include data sources such as wearable sensors, removing the need to insert an additional wire into the patient circulatory system.

Preferably, the functional based metrics may be selected from a group including, but not limited to: Virtual microvessel function (vMF); Virtual ejection fraction (vEF); Virtual pulse wave velocity (vPWV); Virtual arterial distensibility; Virtual augmentation pressure; Contrast pooling; Virtual vessel strain (VVS); and subsequent derivations of these metrics including transient changes over one or several cardiac cycles and as outlined in the illustrated embodiments.

Preferably, metrics may be derived from intravascular imaging, including but not limited to: Artery wall properties (i.e. stiffness, Young's modulus and nonlinear material coefficients); Stent strut malapposition; inflammatory or biological responses; and subsequent derivations of these metrics from the illustrated embodiments or various intravascular catheter systems (i.e. from near-infrared fluorescence).

Preferably, the fluid mechanics-based metrics may be selected from a group including, but not limited to: Pressure drop; Wall shear stress; Velocity; Helical flow; and subsequent variations of these metrics including: Wall shear stress gradient; Transverse wall shear stress; Cross flow index; Axial shear stress; Secondary shear stress; Wall shear stress divergence; Critical point properties;

Wall shear stress exposure time; H1 to H4 helical flow; and their variation over one or several cardiac cycles.

The fluid mechanics-based metrics may further be selected from a group including:
Invariant manifolds;
Lagrangian coherent structures;
Ratio of intraluminal flow to area;
Ratio of intraluminal flow imbalance;
Turbulent kinetic energy; and
Fluid strain rate.

Preferably, the solid mechanics-based metrics may be selected from a group including, but not limited to: Displacement; Principal stress; Principal stress gradient; Principal shear; Principal strain; Tensor divergence; and subsequent derivations of the Cauchy stress tensor including transient variations over one or several cardiac cycles.

The solid mechanics-based metrics may further be selected from a group including:
Structural axial shear magnitude;
Structural secondary shear magnitude;
Structural radial shear magnitude;
Axial Principal stress magnitude and normalised misalignment (from the axial vector);
Secondary Principal stress magnitude and normalised misalignment (from the secondary vector);
Radial Principal stress magnitude and normalised misalignment (from the radial vector); and Invariant manifolds.

Preferably. metrics may further be selected from available patient characteristics including but not limited to clinical presentation or clinical notes and lifestyle factors such as: Stable or unstable patients; ST elevation myocardial infarction (STEMI); non-ST elevation myocardial infarction (NSTEMI); Myocardial infarction non-obstructive coronary arteries (MINOCA); Occluded vessel(s); ECG factors; Heart rate; Blood pressure; Troponin; Cholesterol; Smoking status, body mass index; and sex.

In a preferred form, the method steps are contained within an algorithm of a software program. Therefore, in another aspect of the invention there is proposed a software program for implementing at least some of the steps of the above method.

The software program may be implemented as one or more modules for undertaking the steps of the present invention on a computer system. The modules can be packaged functional hardware units for use with other components or modules. The reader will appreciate that multiple central processing units (CPU) or graphical processing units (GPU) may be used to undertake the steps of the method either at a single or several geographical locations or in the cloud.

Relevant application software may be stored in a computer readable medium such as electronic, magnetic, optical, electromagnetic, infrared, or semiconductor system, apparatus, system, device, propagation medium, or computer memory. In one possible embodiment, the system described herein includes hardware coupled to a microprocessor, microcontroller, System on Chip (“SOC”), or any other programmable device.

In still another aspect of the invention there is proposed an apparatus for implementing any aspect of the above method. The apparatus may include embedded software or firmware with corresponding hardware that is designed to perform one or more dedicated functions.

The apparatus may also include a processor/s and a memory component/s, wherein the data is temporarily stored in the memory component/s, before it is transmitted at predetermined intervals or is interrogated by a device to retrieve the data. The memory component/s may be non-volatile, flash or cache storage device/s.

The processor/s and the memory component/s cooperate with each other and with other components of a computer or computers to perform the functionality described herein. Some of the functionality described herein can be accomplished with dedicated electronics hardwired to perform the described functions.

Communication between the components of the apparatus may be by way of long-range or short-range networks, such as but not limited to low power radio network, microwave data links, 3G/4G/5G telecommunications networks, BLUETOOTH®, BLUETOOTH® Low Energy (BLE), Wi-Fi, LoRa™, NB-IOT, Ethernet, Fibre channel (FC), other types of wired or wireless network, or be connectable to a device that utilises such network/s.

Some of the components of the system may be connected by way of a communication device such as, but not limited to, a modem communication path, a computer network such as a local area network (LAN), Internet, or fixed cables.

Some aspects of the system may communicate in real time via aforementioned systems for processing of one or more modules at one or more physical location(s) while users are interacting with one or more module(s) at another physical location(s). The apparatus may utilise cloud servers and may include embedded software or firmware with corresponding hardware that is designed to perform one or more dedicated functions of the present invention.

The designated software program may alternatively be stored in a computer readable medium on a storage device such as a hard drive, a magneto-optical disk drive, CD-ROM, integrated circuit, a radio or infra-red transmission channel between the computer and another device, a computer readable card such as a PCMCIA card, a flash drive or any other of the number of non-volatile storage devices as either standalone devices or as part of a dedicated storage network such as storage area network (SAN).

The foregoing is merely exemplary of relevant computer readable mediums. Other computer readable mediums may be practiced without departing from the scope of the invention.

### BRIEF DESCRIPTION OF THE DRAWINGS

The accompanying drawings, which are incorporated in and constitute a part of this specification, illustrate an implementation of the invention and, together with the description and claims, serve to explain the advantages and principles of the invention. In the drawings,

FIG. 1 is a flowchart of the system for providing an evidence-based prognosis/prediction and visualisation to a clinician;

FIG. 2 is a node-based flowchart illustrating the process at each node (i.e. clinic/hospital);

FIG. 3 is a flowchart of the centralised cloud compute or containerised instance for carrying out detailed analytics based on the data acquired from each node;

FIG. 4 is a schematic of exemplary computer hardware and/or systems on which the enclosed embodiments are processed;

FIG. 5 is a schematic of the intravascular machine learning approach to segment various features while overcoming limitations within the imaging system;

FIG. 6 is an outline of invasive coronary angiography (ICA) acquisition properties and preprocessing relevant to the enclosed embodiments;

FIG. 7 is a schematic of the machine learning workflow to segment and reconstruct the three-dimensional vasculature via an angiographic neural radiance field (ANeRF);

FIG. 8 illustrates the process to process transient information from invasive angiography including for virtual assessment of ventricle function;

FIG. 9 is a schematic of the multi-level segmentation possible from the preceding embodiments;

FIG. 10 is a schematic view of a blood vessel showing contrast flow and dissipation properties relevant to assessing microvasculature and/or functional properties from angiogram images in the disclosed embodiments;

FIG. 11 is a flowchart of the process to quantify microvessel function in invasive coronary angiography without an additional invasive wire and one of its applications in augmenting boundary conditions for generalised metric assessment;

FIG. 12 illustrates a flowchart for the co-registration or augmentation of multiple imaging modalities into a single spatio-temporal model for both analytic and visualisation purposes;

FIG. 13 illustrates the selection of data or features for the ‘level one’ and ‘level two’ analyses;

FIG. 14 is a schematic of logging multiple events or data inputs over time for a single patient within the proposed embodiments;

FIG. 15 is a schematic of the machine learning decision making approach;

FIG. 16 is an example overview of the intravascular imaging visualisation and user interface;

FIG. 17 is a further example of the simplified user interface with predictive and demographic comparisons;

FIG. 18 is exemplary of the augmented user interface containing data from multiple modalities, analytics and/or predictive results in a single interface; and

FIG. 19 illustrates an example of indicative performance.

### DETAILED DESCRIPTION OF THE ILLUSTRATED AND EXEMPLIFIED EMBODIMENTS

Similar reference characters indicate corresponding parts throughout the drawings. Dimensions of certain parts shown in the drawings may have been modified and/or exaggerated for the purposes of clarity or illustration.

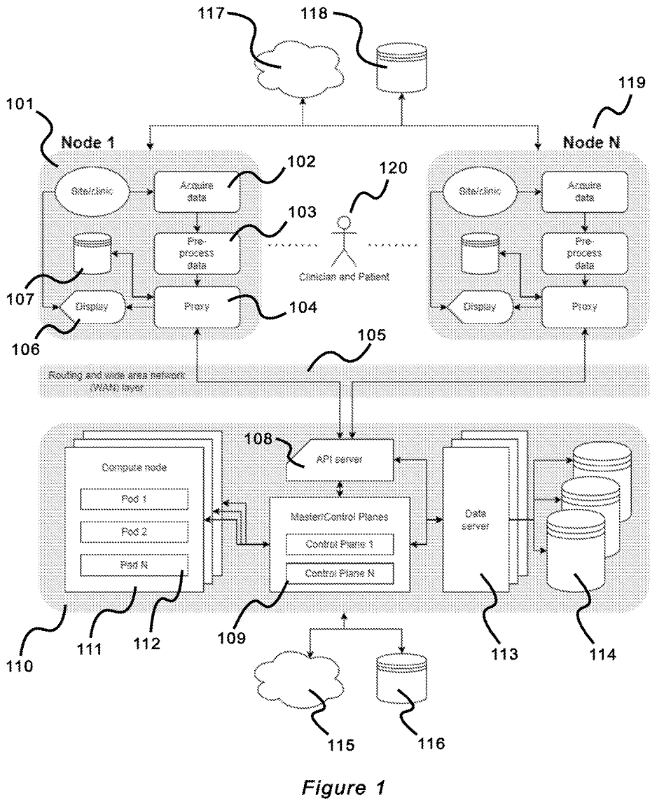

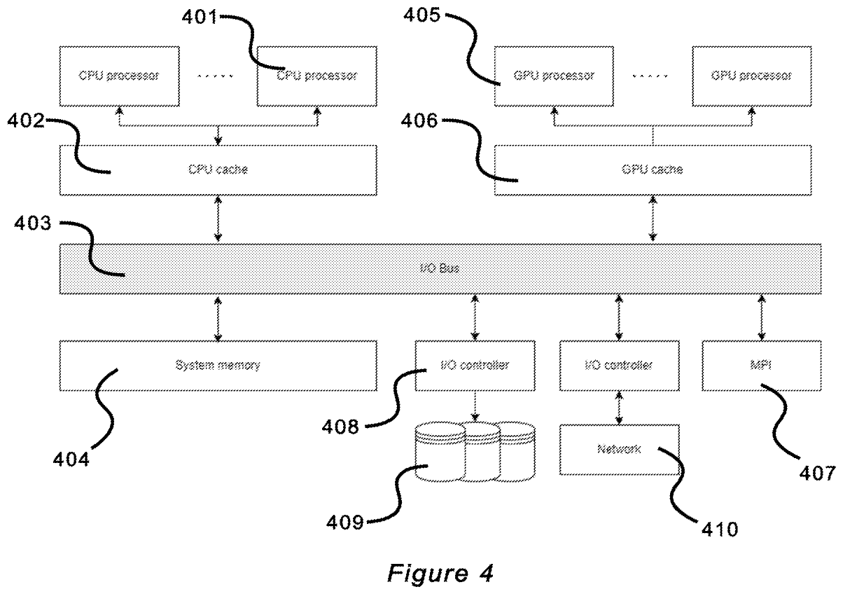

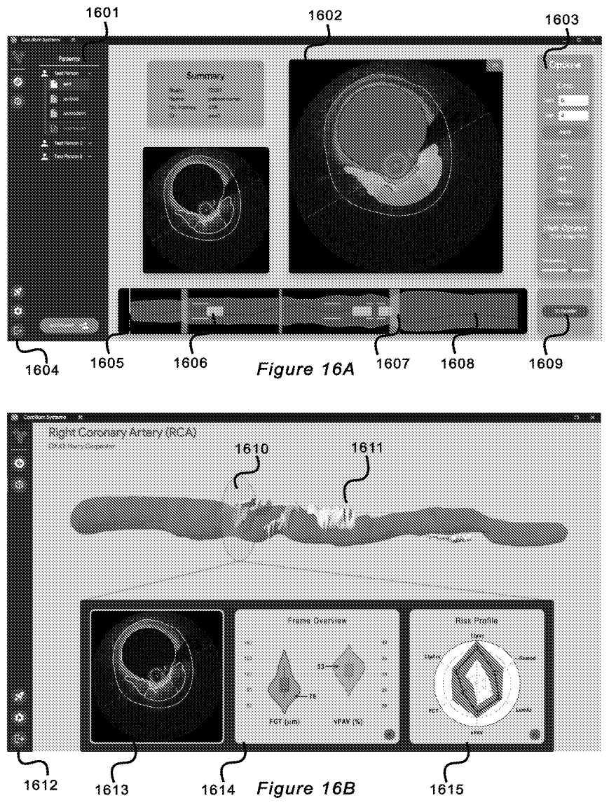

Turning to FIG. 1, a computer system is defined in at least some embodiments to implement the enclosed methods of producing a predictive model of the artery/vasculature of a patient to present a risk analysis to predict future changes in coronary disease and suggests optimal treatment pathways based on artificial intelligence and biomechanical simulations. The flowchart illustrates Node 1 [101]-Node N [119] as any number of connected nodes which are independent sites (such as clinics/hospitals) and may operate individually or as connected services and may or may not be connected to various third-party cloud [117] or data systems [118] such as patient archiving and communication (PACS) system(s). Within each node, data is acquired from a patient or patients from local data sources [102] or from the connected third-party cloud [117] or data systems [118]. The data is pre-processed [103] on a compute device or devices and associated hardware of which embodiments are outlined in further detail in FIG. 4 and on which dedicated software program or programs may preferably exist in local or cloud-based forms. Such pre-processing may preferably include quality checks (which may comprise missing or null data entry handling, image visual quality assessment and filtering or modifying in various cases, metadata extraction and logging and/or de-identification). This data may preferably be transferred via a proxy server [104] over a dedicated wide area network (WAN) [105] to the centralised compute instance(s) for further analytics. The communication via the node to the centralised compute instance may also be carried out over various other communication media or networks. The pre-processed data may also preferably be displayed [106] locally at the node through various display hardware, firmware or dedicated technologies using dedicated software enclosed embodiments from FIGS. 16, 17 and 18. The pre-processed data may also be passed directly or via proxy server(s) to data storage media with locally [107] or cloud based [117 and 118].

The centralised cloud compute system will receive pre-processed data preferably via the WAN but also via other network interfaces and communication protocols. Data is received via an application programming interface (API) server [108] which may be a dedicated server or form part of the master/control nodes [109] which themselves consist of preferably three or more control planes for provision of a high-availability (HA) cluster service. The master/control plane and or API server may preferably validate incoming data and prepare or configure object instances such as through container management systems including Kubernetes for compute nodes [111], pods [112] and other service component level interaction. In one embodiment a compute node may communicate with the master/control plane and take instruction to run a pod (a computer program or set of instructions) via different levels of general hardware (see FIG. 4). In another embodiment the compute node may communicate with the master/control plane and take instruction to run several concurrent or parallel pods on one or several compute nodes. In another embodiment the management system(s) or compute platform(s) may include Docker, OpenShift, Amazon Web Services, Microsoft Azure and associated variations to manage and run pod or container-based instances and pipelines. Master/control plane(s) and or API server(s) may preferably also communicate between compute nodes, WAN and preferably data server(s) [113] running storage area networks (SA) that can be configured to include volatile, non-volatile or flash memory technology and variations of electronic data storage devices [114]. The centralised compute system may also communicate via various protocols with third part cloud [115] or data storage systems [116]. The centralised compute instances and SAN's are accessible from authenticated nodes where users including clinicians, technicians and patients [120] can access and visualise data, results, reports and instruct the system to carry out further processes.

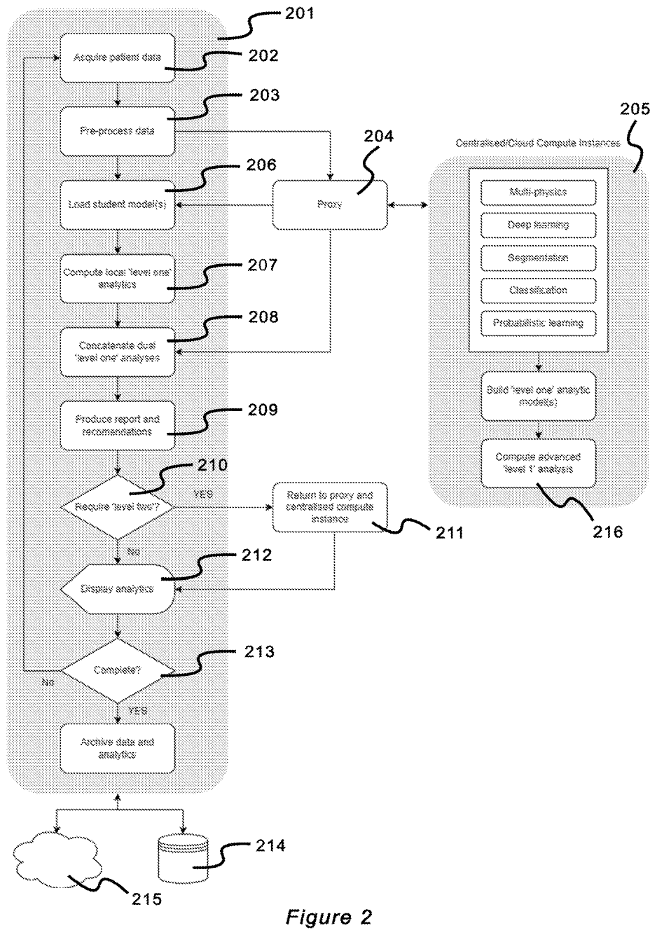

FIG. 2 illustrates a node-based flowchart setting out the process at each node (i.e. clinic/hospital) [201]. Preferably data is acquire from the local electronic network or connected third part of cloud based systems patient-specific data including but not limited to structural, functional or chemo-biological imaging, blood pressure/velocity/catheter-based measurements, presentation (which may include ST-elevation myocardial infarction [STEMI], non-ST elevation myocardial infarction [N-STEMI], myocardial infarction non-obstructive coronary arteries [MINOCA]) and various other clinical notes and manual inputs from experienced technicians or clinicians such as stable or unstable patients [202]. Acquired data is pre-processed to handle ambiguity, noise and missing data values using a compute device or devices and associated hardware of which embodiments are outlined in further detail in FIG. 4 and on which dedicated software program or programs may preferably exist in local or cloud-based forms [203]. Such pre-processing may preferably include quality checks including but not limited to missing or null data entry handling, image visual quality assessment and filtering or modifying in various cases, metadata extraction and logging and/or data de-identification. In parallel and in one embodiment the pre-processed data is passed via a communication network or WAN of one or various protocols via a proxy server to the centralised or cloud compute instance [205]. Also communicated via the network or WAN and proxy server to the centralised or cloud compute instance are details on the local hardware capability and usage including but not limited to parallel processor number, system and cache memory, graphics processing unit number and detail on graphics or tensor cores and graphics cache and various associated embodiments.

In another embodiment and in parallel to the previous step the processed data is passed to a student machine leaning model [206] whose features and/or design and/or weights are pulled via a machine learning operations (MLOps) pipeline from the proxy server [204] and the centralised or cloud compute instance [205]. The student model may preferably be modified by a teacher model to optimise or meet local hardware requirements that were passed to the proxy server and centralised/cloud compute server through previous steps. The student model then carries out a ‘level one’ analysis [207] on local general hardware (expressed here as the ‘local level one’ analysis) and whose hardware features and protocols are outlined in FIG. 4 and may include general purpose central compute processors or graphic processing units or accelerators to deliver a real-time analysis. Simultaneously, an ‘level one’ analysis is carried out on the centralised or cloud compute instance [205] which is expressed here as an ‘advanced level one’ analysis and is preferably optimised to deliver all or some analytic results not possible on local hardware within the required timeframe (i.e., in near-real time) [216]. This ‘advanced level one’ analysis is communicated via the proxy server to the local node [201] and concatenated with the ‘local level one’ analysis [208]. In another embodiment the entire ‘level one’ analysis may take place via the centralised or cloud compute instance if local hardware or firmware requirements do not provide sufficient processing capability. In another embodiment the entire ‘level one’ analysis may take place locally. It should be appreciated that such an approach is designed to optimise available hardware resources as described in FIG. 4 across multiple sites or locations. The level one analyses may also preferably include the steps of: processing one or several two-dimensional images; generating a three-dimensional map of the vasculature and its static and/or transient anatomy; calculating via various embodiments a set of metrics; and using the set of metrics to provide one or several analytic and predictive models.

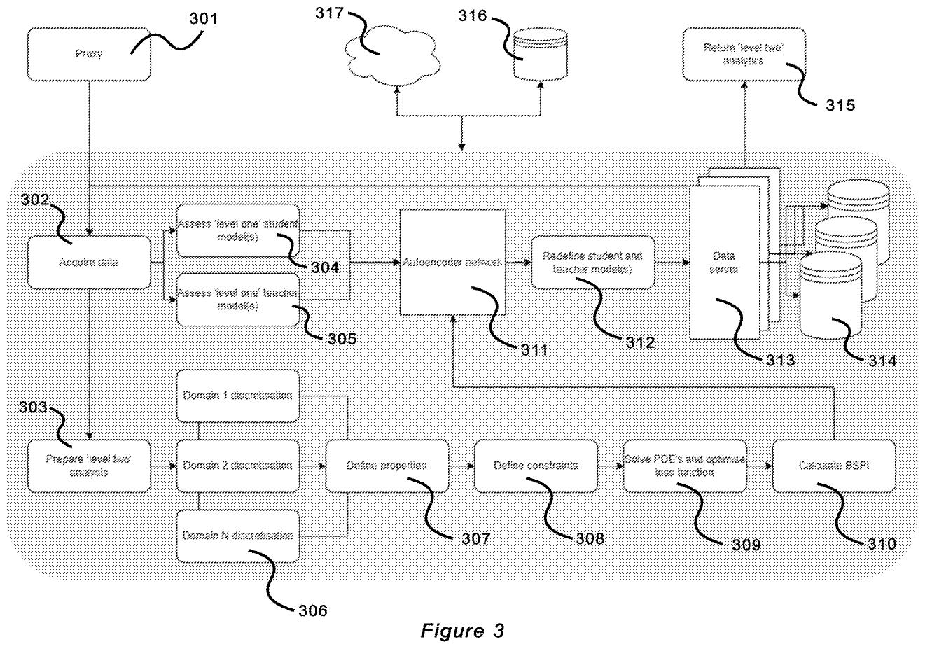

After concatenation of ‘level one’ analysis a report and recommendations is produced [209] and a decision on the need for a more advanced analytic ‘level two’ simulation is made [210]. The ‘level one’ analysis may preferably suggest or recommend the need for a ‘level two’ analysis or the decision may be made by an experienced user. Upon decision to carry out a detailed ‘level two’ analysis the pre-processed data and ‘level one’ analysis is passed via the electronic network and the proxy server to the centralised or cloud compute instance for processing [211]. This step is detailed further in FIG. 3. If no the results are visualised or displayed [212] for the user as described in the enclosed embodiments. Preferably this display could include ‘level one’ or ‘level two’ analyses or both and the associated metrics or visualisations in multiple dimensions. In another embodiment this display preferably includes data from one or more imaging modalities which may be different types of imaging and are augmented into a single user interface and display. If the analysis is complete [213] the data is archived or stored on local electronic storage media or third-party cloud [215] and data storage systems [214] which can be accessed at any future stage by authenticated user or patients. If the analysis is incomplete the process can proceed to acquiring further data [202].

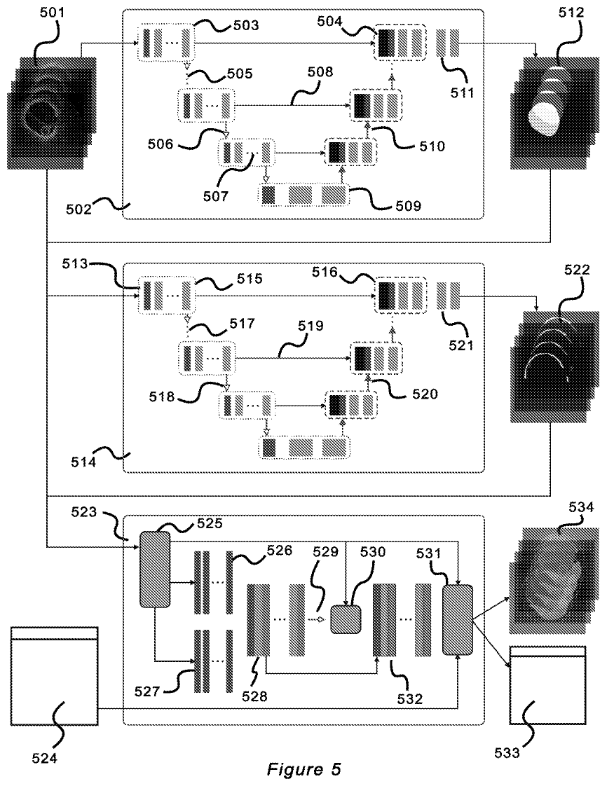

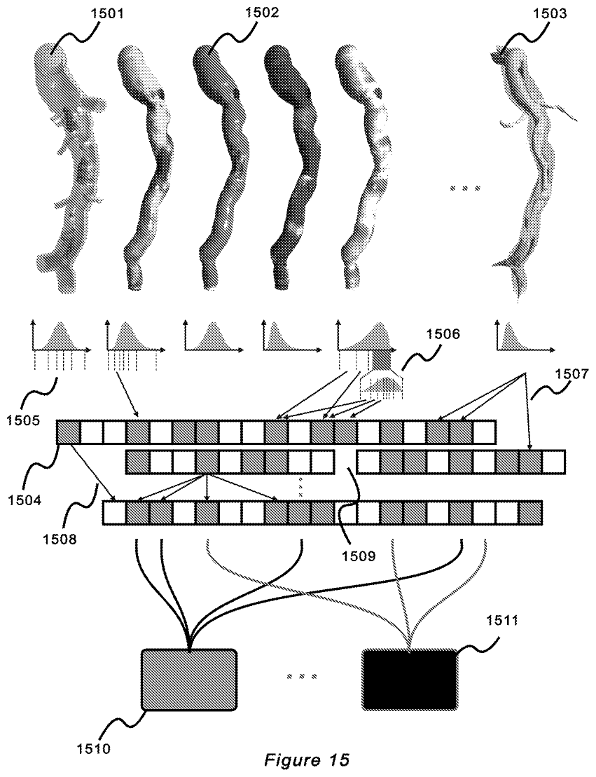

FIG. 3 is a flowchart of the centralised cloud compute or containerised instance and the process of carrying out detailed analytics based on the data acquired from each node. The centralised cloud compute or containerised instance(s) may preferably connect through a proxy server [301] to one or multiple nodes simultaneously and acquire data [302] from the embodiments enclosed in FIG. 2 including pre-processed data and ‘level one’ analyses. Two parallel operations are carried out on one or more pods or compute nodes and associated generalised hardware and firmware components upon scheduling by the master/control node. First, a ‘level two’ analysis is prepared [303] from the acquired node-based data and from data servers [313] and electronic storage medium [314] that may preferably include pre-trained machine learning model features or weights and experimental multi-physics and physiology laws. Information from structural imaging features including three-dimensional models in various forms including three-dimensional image stacks, three-dimensional density fields, three-dimensional adaptive mesh or three-dimensional point clouds is first discretised by domain [306]. In one embodiment domain discretisation may preferably include the process of dividing the features into finite element or finite volume elements. In another embodiment boundary intersections or contact regions may be calculated with other mesh-based descriptions of three-dimensional features across broadly associated fields of fluid mechanics, structural mechanics, electro-mechanical coupling, structural-fluid coupling and chemo-mechanical coupling. The discretised domain properties are applied [307] based on experimental multi-physics or physiology such as estimated nonlinear tissue properties extracted from imaging modalities (see exemplary embodiment in FIG. 5) which is considered just one exemplary case. Constraints [308] such as boundary or initial conditions are also defined based on acquired data which may preferably include measured or input data points from each specific patient or may also include augmented data if null or missing inputs are detected from previous embodiments. After defining constraints the partial differential equations are solved in one embodiment by the finite element or finite volume techniques or in another embodiment by a neural network and an associated loss function [309] to produce preferably one or more metrics. The calculated metrics are then used to determine a unique biomechanical stress profiling index (BSPI) [310] which preferably uses all metrics to identify an outcome. In another embodiment the BSPI uses a subset of metrics chosen using embodiments described in FIG. 15 to identify or predict one or several independent or linked outcomes. In parallel to this process the student [304] and teacher [305] machine learning model are assessed independently and passed into a deep variational autoencoder network [311] along with results from the previous BSPI analysis and stored data from local or cloud data server databases. The autoencoder network preferably performs unsupervised lower dimensional latent representation of the detailed ‘level two’ analyses and uses error or variance with the teacher and student models to rebuild a detailed teacher model and lightweight student model suited to the local node-based hardware or firmware requirements. In another embodiment the autoencoder may only receive the student and teacher models and instead performs a federated learning optimisation using features of the local node-based student model and the global teacher model without passing patient details to re-optimise or rebuild the local-student model from the teacher model again suited to the local node-based hardware or firmware requirements. The completed model(s), feature(s), trained weight(s) or other data is then passed through the data server [313] and associated networks and storage medium [314]. Relevant ‘level two’ analytics are preferably passed vie the proxy server and communication networks back to the node for interactive visualisation by the user.

In various embodiments the systems and methods may preferably be implemented on or using general purpose computing components illustrated in FIG. 4. In one embodiment the computing components may include a central processing unit (CPU) [401] with varying levels of processor cache [402] which is coupled via the input/output (I/O) bus [403] to system memory [404]. In another embodiment the computing components may also include a graphical processing unit (GPU) [405] or acceleration component such as a tensor processing unit with varying levels of graphical cache and memory [406] that communicates through the I/O bus [403] with system memory [404] and other system components. System memory may preferably be configured to store data or code for rapid access to CPU(s) and GPU(s)/accelerator(s) and be configured to include volatile, non-volatile or flash memory technology and derivations of such technology. The components may also include an I/O controller [408] with access to internal or external electronic storage media [409] and/or networks and connected devices in various formats [410]. In one such format wired or wireless data communication to storage networks may include Ethernet or Fibre Channel (FC), low power radio network, microwave data links, 3G/4G/5G telecommunications networks, BLUETOOTH®, BLUETOOTH® Low Energy (BLE), Wi-Fi, LoRa™ and NB-IOT communications to computer readable storage medium such as electronic, magnetic, optical, electromagnetic, infrared, or semiconductor system, apparatus, system, device, propagation medium, or computer memory componentry arranged and managed by data server(s) in storage area networks (SAN) that may preferably include the aforementioned hardware

In yet another embodiment the computing components may contain a single or multiprocessor CPU [401] system or varying or identical architectures consisting of several processors capable of executing instructions or calculations relating to the enclosed embodiments. In one embodiment multiprocessor components may communicate through message passing interfaces (MPI) [407] which may also preferably communicate between servers each containing single or multiple CPU processors via various communication or network protocols. In other embodiments other message passing protocols may be used for parallel processing of instructions on one or several processors and/or servers. The computing components may also contain one or several GPU or acceleration devices [405] of varying or identical architectures to carrying out instructions and may similarly communicate between devices and servers with one or multi-GPU/accelerator components via various communication or network protocols.

FIG. 5 is schematic of the intravascular machine learning approach to segment various features while overcoming limitations within the imaging system including but not limited to limited tissue penetration depth of the imaging system, susceptibility to artefacts including residual blood from improper clearance and rotational distortion. The intravascular imaging pullback [501] is acquired as two-dimensional slices stacked axially with the entire stack of acquired images passed into a spatio-temporal U-Net machine learning architecture [502] that leverages long-short term memory (LSTM) and attention mechanisms for robustness and generalisation strength in sparse and noisy real-world data. The encoder modules [503] visualised in the down-sampling aspect of the architecture build on a modified ResNet backbone to incorporate three-dimensional blocks to improve continuity of segmentation across sequential two-dimensional imaging slices. Vertical [505] dynamic layering enables architecture adaptation to datasets overcoming limitations of static network designs. Automated adaptive block contraction [507] further improves feature extraction and generalisation in changing datasets by altering convolution blocks at each level. Three-dimensional max pooling [506] in the encoder downsamples the feature maps while three-dimensional upsamples the features maps via trilinear interpolation [510] in the decoder module. Traditional skip connections correspond to the number of dynamically provisioned layers [508]. Three-dimensional, temporal convolutional decoder blocks [504] are built of dual three-dimensional convolution, three-dimensional batch normalisation and a rectified linear activation function passed into a single long-short term memory layer with visibility of the entire image stack, and attention mechanism and a final activation function. The output segmentation map [511] produces masks of the lumen including branch regions throughout the imaging stack [512].

The lumen segmentation map is used to mask the input image stack [513] to a modified three-dimensional DenseNet based decoder architecture [514] to identify visible components of the medial layer. Three-dimensional decoder blocks [515] use the same vertical dynamic layering [517] and three-dimensional max pooling [518] as the previous model with similar cross-dense connections [519] and concatenation in the decoder blocks [516] which expand the receptive field through large dilation and consist of dual three-dimensional convolution, three-dimensional batch normalisation and leaky rectified linear activation functions for improved segmentation of small but important and noisy features of the medial layer. The output layer [521] produces a binary stack which is applied with the lumen segmentation map and the original image stack for use in the final stage.

The original image stack [501], lumen segmentation map [512] and medial binary mask [522] are passed as inputs to a preprocessing block [525] before application to a modified deep physics informed neural network architecture [523]. The preprocessing block determines the mask-based centroids to produce a smoothed vessel centerline (as opposed to the catheter centroid which is located at the image center) and then feeds three-dimensional pixel coordinates [526] and associated pixel colour data and segmented lumen and visible medial layer maps [527] as inputs to stage one of the modified physics informed neural network. These inputs are concatenated and fed into the stage one multi-layer perceptron(s) [528] consisting of fully connected layers with activation and batch normalisation before max pooling [529] to produce a global feature set [530] from the image stack and segmentation maps. The global feature set also draws specific features that can be identified directly from the preprocessing block (i.e. chosen algorithmically or by an experienced user before automated processing) such as the lumen centroid in each frame. Local features [528] are also fed forward and concatenated with the global feature set before the second stage multi-layer perceptron(s) [532]. Pre-defined partial differential equations [524] governing tissue continuity, nonlinear tissue properties, imaging properties (including spectral, frequency and time domain optical properties in the case of optical coherence tomography imaging systems) combine with blood pressure measurements and spatial derivatives from the multi-layer perceptron(s) and pre-processed information from the lumen and visible media [525] to produce the customised loss function. The latter provide further initial and boundary conditions that improve convergence. The customised loss function may preferably backpropagate features throughout the network to constrain the pixel/image-based network segmentation with knowledge of the imaging system and vasculature physics. In another embodiment the pre-defined partial differential equations [524] may impose physics related to one or several imaging systems and it should be appreciated that the embodied method may be applied to other intravascular imaging systems without departing from the scope of the invention. The output is a segmentation map including plaque components and vessel structure in attenuated areas [534] suitable for various voxel or density based three-dimensional reconstructions and an estimated tissue property map [533].

The entirety of the method is carried out on a graphical processing unit (GPU) after taking the imaging stack and associated physiological data as inputs from system memory. Four outputs are produced including the lumen segmentation, visible media layer segmentation, outer adventitial wall and plaque component segmentation and associated estimated tissue properties which are communicated back to system memory from video memory upon completion of the process. Inter process communication between lumen, visible medial and advential/plaque segmentation and fitting is passed through video cache while the original image stack is stored in video memory for rapid processing as a single operation of which the general hardware components are described in FIG. 4.

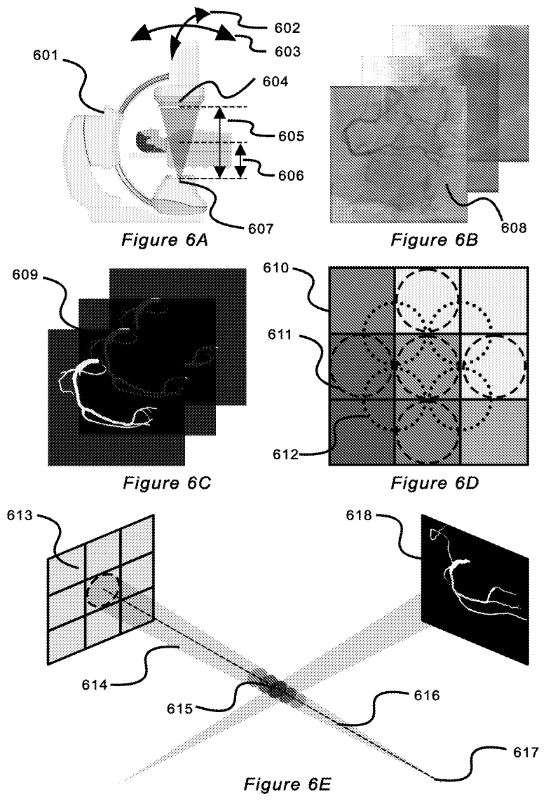

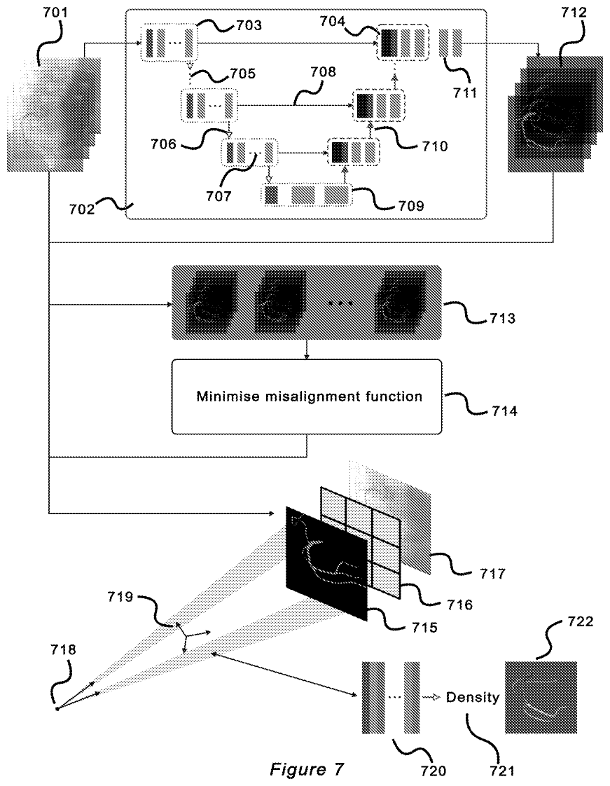

FIG. 6 outlines the key features of invasive angiography and our associated embodiments. Turning to FIG. 6A, properties of the angiographic C-arm machine [601] considered useful for subsequent embodiments are defined including primary [602] and secondary [603] angles, location and spatial properties of the detector module [604], location of the X-ray source point [607], distance from the source to the c-arm isocenter [606] and distance from the source to the detector module [605]. The detector module captures x-ray properties to produce an image sequence of vessel structure over the cardiac cycle(s) [608] where vascular structures are illuminated through injected contrast and may not be visible over the entirety of the image sequence. Machine learning or numerical approaches are used to identify vascular structures throughout the image sequence and produce binary maps [609] (see FIG. 7 for further detail on these embodiments). It should be appreciated that these binary maps are used to enhance vascular structures in the subsequent processing steps. Unlike previous approaches that try to purely identify vascular structures or remove/filter the background noise, our approach still utilises this background noise in a novel embodiment of the subsequent illustrations. Turning to FIG. 6D, the binary map is used to produce a transient multigrid across the vessel regions and its boundaries by taking the original image sequence [610], pixel locations [611] and pixel boundaries [612] which are used in the construction of the angiographic neural radiance field with spatial structure illustrated by FIG. 6E. It should be highlighted the choice of circular domains at pixel [611] and pixel intersections [612] is the frustrum of the cone beam [614] that is used to represent x-ray projections to the plane, rather than typical single-beam ray projections [616]. The c-arm inputs [602-607] are used to orient the angiographic multigrid representation [610] in three-dimensional space [613]. The described method may preferably take at least one angiographic plane as input with metadata on gantry orientation and may produce or render subsequent optimal two-dimensional projections [618] or the vasculature to assist in patient assessment or decision making or further algorithm development. In another embodiment the method may take several input planes [613 and 618] to generate the three-dimensional density map. Unlike pinhole camera models which place the three-dimensional scene to be rendered beyond the image plane (so that a light ray emitted from the camera passes through the image plane and is then projected to the near and far-field of the camera view and which can then be sampled), invasive angiography locates the ‘scene’ (patient) between the source (x-ray) [607 and 617] and the imaging plane (detector) [604 and 613] where the resulting image can be thought of as a ‘shadow’ driven by tissue or contrast density (so an X-ray is emitted from the detector and first passes through the ‘scene’ before being captured by the detector requiring a redefinition of the sampling strategy and an inability to capture visible radiance or colour). At or near the isocenter of the c-arm system the cone beam contains integrated positional, size and density encodings [615] in the c-arm gantry coordinate system.

FIG. 7 is a schematic of the machine learning workflow to segment and reconstruct the three-dimensional vasculature via an angiographic neural radiance field (ANeRF). In one embodiment the process may also be considered as an angiographic neural impedance field (ANIF) as the process of generating the density map from a C-arm gantry coordinate system is done from X-ray absorption between the source and detector plane (hence impeding the passage of X-rays and producing an effective shadow). In one embodiment the first stage of angiographic processing involves segmenting the temporal image stack [701] to produce a binary mask of vascular structures [712]A modified U-Net architecture [702] is used with the stack input to a modified temporal DenseNet based encoder [703] to identify vascular structures illuminated by contrast. In another embodiment the stack is input to a standard two-dimensional DenseNet architecture for individual image processing. In yet another embodiment individual images may be processed with numerical processes such as a Frangi vesselness filter. In the temporal embodiment the Three-dimensional decoder blocks [703] use the same vertical dynamic layering [705] and three-dimensional max pooling [706] as the previous model with similar cross-dense connections [708] and concatenation in the decoder blocks [704] which expand the receptive field through large dilation and consist of dual three-dimensional convolution, three-dimensional batch normalisation and leaky rectified linear activation functions for improved segmentation of small but important and noisy features of the medial layer. The output layer [711] produces a binary stack [712] which is applied with the lumen segmentation map and the original image stack for use in the final stage.

If more than one angiographic image stack is available [713] for either the left or right vasculature, a pre-processing step is first implemented to co-register the stacks and overcome imaging system artefacts and misalignments that frequent invasive angiography systems. The co-registration may preferably use identifiable features such as branch regions between image stacks to minimise a misalignment function [714] which aims to minimise the distance between each set of ray-tracing projections between identified feature(s). The function takes these feature locations and C-arm gantry orientations as input and introduces a scaling factor, x′ and y′ detector misalignment distances for each view as well as a global vector for C-arm source-detector misalignment. In another embodiment the misalignment function may include a transient morphing factor which uses second order interpolation to bridge the large temporal gaps between angiographic frames and shifts features forward or backward in time to morph the identified image frame to better handle ambiguities in cardiac gating. The minimisation function may preferably be solved by general purpose optimisation algorithms and returns a set of offset corrections to be applied to each angiographic view. The original image stack [717] and its multigrid representation [716] and processed binary stack [715] are then passed as inputs to the angiographic neural radiance field along with information on the pixel scaling properties (i.e. detector sizing), primary and secondary orientations, source to isocenter and detector distances and offset corrections (if available from the pre-processing step). Unlike traditional neural radiance field approaches and their subsequent derivations, the embodied angiographic neural radiance field (ANeRF) does not encode knowledge of colour but only extracts three-dimensional density [721] from its multilayer perceptron(s) while inverting the order of projections to handle scene location [719] between source [718] and detector planes [717].

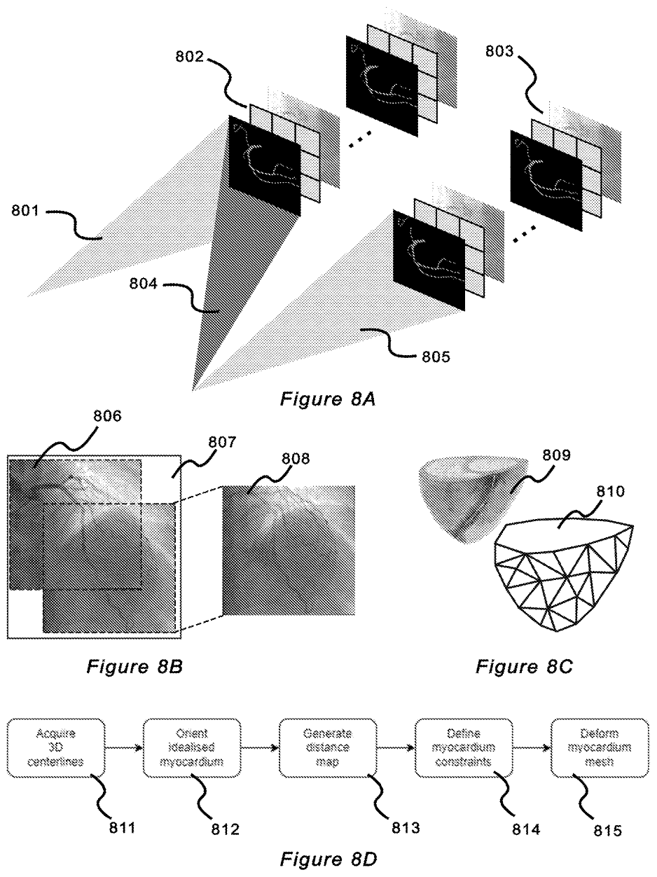

FIG. 8 illustrates the steps in leveraging features of the previous ANeRF embodiment to improve both transient analyses of the vascular and ventricular reconstruction to enable virtual assessment of ventricle function either with or without additional ventriculography. As a ventriculogram is performed in as little as 50% of invasive angiographic procedures and requires additional radiation and procedure time, the ability to extract similar characteristics without it is important for clinicians. Turning to FIG. 8A the transient nature of invasive angiography and limited angiographic view windows means acquisitions contain significant motion artefacts. To handle cardiac, table and detector motion, each frame across an image stack is processed using the previous embodiments to identify vascular structures. The stack of processed data is then orientated with the X-ray source projection [801] to the detector location with subsequent segmented mask(s), original image(s) and multigrid representation(s) [802]. Co-registration or motion adaption may result in rigid body transformation of the entire system [805]. In other cases, the detector may be shifted to view a wider region of the vasculature requiring a re-orientation of the coordinate system for the new detector-source projection region [804] in each frame of an acquisition. Images throughout each transient stack [802 and 803] are then registered to various states of the cardiac cycle and the varying c-arm gantry coordinate system such that frames may preferably be ‘stitched’ together to widen the effective view of each individual frame to the entirety of the region covered during detector or c-arm gantry motion. Tuning to FIG. 8B, this ‘stitching’ is illustrated whereby an early frame from an acquisition (e.g. Frame 15) [806] is joined with a later frame from the same acquisition (e.g. Frame 24) that was subject to detector or other motion of the c-arm system or patient [808] to generate a new image region [807]. Frames are stitched together by using the inverse of the segmented vascular structure to instead generate a centrally weighted map of the background tissues throughout each frame. The background tissue structures are centrally weighted to preferably use key features closer to the centre of each image for co-registration as features at or near the image boundaries may not be visible throughout the entire stack due to the motion artefacts. These background tissues such as ribs or spinal bones are then used to orient each frame and account for c-arm gantry or patient related motion artefacts. The previously illustrated embodiment of the ANeRF is then preferably applied across the stitched image stack to generate a differentiable density field of the entire vasculature, something not possible via previous approaches.

Turning to FIG. 8C, with the stitched representations of the vasculature able to visualise the entire vascular structure, even regions that may not fit within a single image view, an idealised surface map of the ventricles [810] is acquired from a demographically adjusted set of volumetric imaging data [809]. In one embodiment the ventricular surface map may represent both the left and right ventricles. In another embodiment the surface map may represent only the left ventricle. If left ventriculography is available or has been carried out, the cross-sectional anatomy of the ventricle may also be used to adapt the surface mesh to better fit the imaged ventricle. In another embodiment, non-invasive imaging such as echocardiography or computed tomography data on the ventricle structure may be acquired from patient data or third part sources such as the PACS system and used to augment the surface map of the ventricle. Turning to FIG. 8D, a flowchart of the process to co-locate the ventricular surface map to the three-dimensional vasculature model is presented. From the three-dimensional density map of the vasculature preferably created using the stitched image frames, the three-dimensional centreline(s) of the entire vasculature is extracted through numerical methods such as volumetric thinning [811]. The ventricle or myocardium surface map is then orientated and resized to minimise a distance function between the surface and the vasculature centrelines [812]. In one embodiment the surface map may have regions weighted to certain epicardial vessels to assist in accurately aligning the centrelines and surface. A distance map is then generated for equally discretised regions of the centerline(s) [813] to identify the distance between the desired location (centerline) and the closest current location of the surface. In one embodiment the surface may be meshed and constraints on the mesh properties such as smoothness or stiffness of the mesh may be applied [814]. The distance map is then iteratively minimised by deforming the ventricle surface map/mesh to fit the vasculature centreline(s) and produce an estimate of the patient's heart surface and ventricle shape [815]. In one embodiment the process may be repeated for multiple stages of the cardiac cycle to produce an estimate of ventricle function over one or several heartbeats. In another embodiment the final deformed ventricle surface/mesh may be further moved/deformed using the transient motion of the vasculature centrelines to produce an estimate of ventricle function over one or several heartbeats. The changing surface map/mesh is then preferably used with information on the vascular function from previous embodiments to produce a real-time virtual ejection fraction, wall motion index and wall strain function(s).

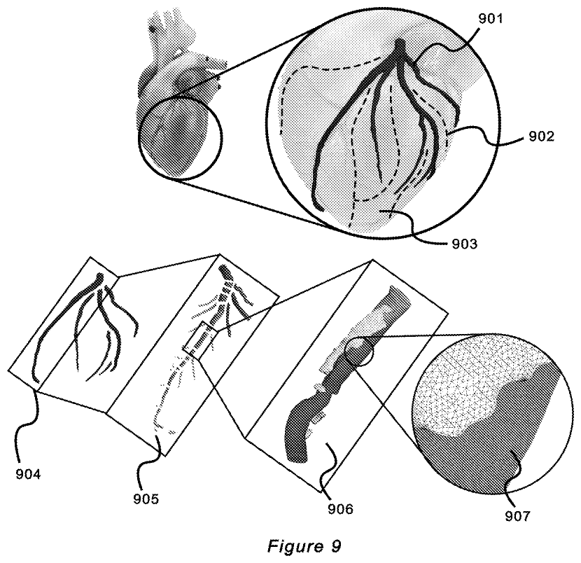

FIG. 9 is a schematic of a multi-level segmentation, in which a description of the multi-level segmentation from major epicardial arteries and myocardium perfusion regions to high-fidelity discretisation of diseased regions for use in simulation or calculations using machine learning decision methods or continuum mechanics methodologies is illustrated and enabled by the previous embodiments. The major epicardial arteries [901] and myocardium [903] are first produced in three-dimensions via the previous embodiments. Perfusion boundaries [902] throughout the myocardium are produced via a three-dimensional region growing approach to produce myocardial sectors associated with each epicardial vessel. Major epicardial arteries are then divided based on bifurcation points [904] into each major epicardial vessel (i.e. left main, left anterior descending, left circumflex, ramus and right coronary artery). Main epicardial arteries are then discretised into sections using minor epicardial vessel branch points (i.e., obtuse marginal, diagonals etc) [905]. Segment-wise anatomy [906] is then discretised for solving via mesh-based techniques [907] such as but not limited to finite element/volume-based continuum mechanics.

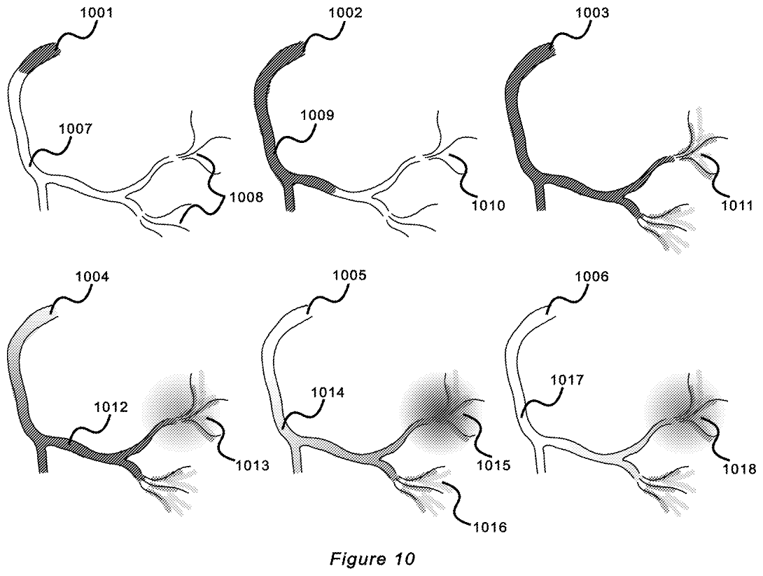

FIG. 10 is a schematic view of a blood vessel, providing a visual indication or description of contrast flow that the illustrated and exemplified embodiments use to assess the microvasculature from invasive angiogram images. The figure illustrates the contrast flow from the epicardial arteries through to perfusion into the myocardium via the micro vessels/microcirculatory system. Administered contrast is first injected through the catheter and begins to flow from the most proximal region [1001] of the left or right epicardial vessel. The remainder of the vessel [1007] and micro vessels [1008] are free of contrast and are generally not visible except in cases of high calcium deposits. As time progresses [1002] contrast travels with blood velocity to fill the epicardial vessels [1009] while the micro vessels are still not visible [1010]. When contrast fills the epicardial vessels [1003] and begins to perfuse into the micro vessels and myocardium which may become visible [1011]. Upon stopping contrast injection blood velocity will drive contrast distally causing contrast dissipation to begin in the most proximal region [1004] while gradually progressing from the epicardial [1012] to micro vessels where abnormal micro-function will lead to increased contrast intensity [1013]. This process continues with time with the epicardial vessel emptying of contrast [1005 and 1014] and healthy micro vessels will see less intense contrast [1016] compared to vessels with abnormal microcirculatory function where contrast can persist in the micro vessels and can be visualised as myocardial blush [1015]. As time progresses visible contrast intensity continues to diminish [1006, 1017 and 1018]. The location, intensity, residence time of the blush and contrast dissipation rate is tracked to identify and grade abnormal perfusion when accounting for three dimensionality of the vessels such as epicardial volume which is extracted via aforementioned embodiments.

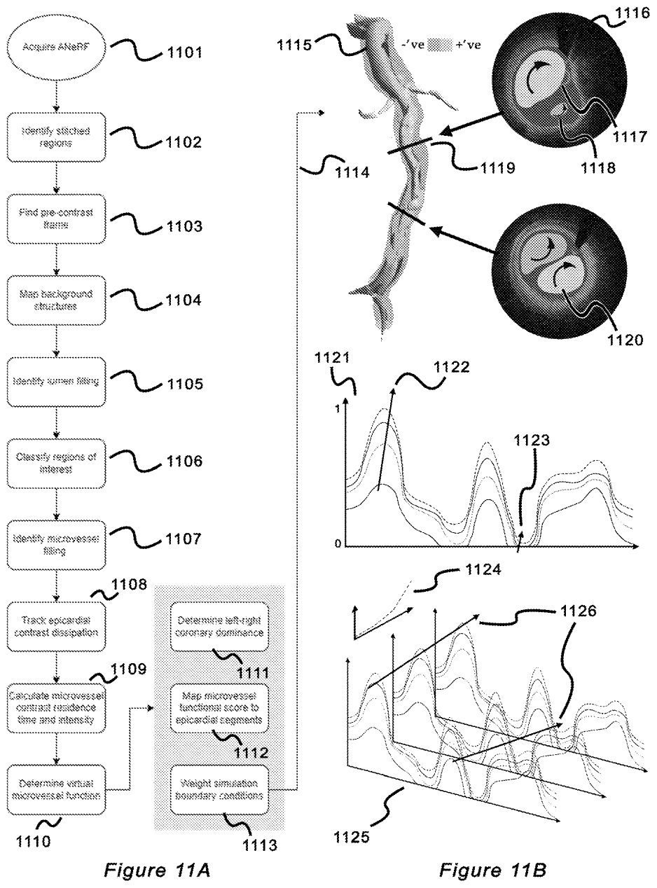

FIG. 11 is a flowchart of the process to quantify microvessel function in invasive coronary angiography without an additional invasive wire and one of its applications in augmenting boundary conditions for generalised metric assessment. Turning to FIG. 11A, the flowchart illustrates the process of using the ANeRF-based process from previous embodiments to determine functional information on the microvessels that feed blood to the myocardium. Beginning with the ANeRF [1101], the stitched regions of the previous embodiments are classified to delineate the regions belonging to different frames. This step allows weighting of vessel contrast to account for the varying time points the stitched images were acquired at. Preferably using numerical or manual methods the frame immediately prior visible contrast being injected into the vessel is determined [1103]. In another embodiment this frame may be selected by gating the frame count to the administration of contrast. Without vascular structures being illuminated by contrast the background structures morphology and density are mapped to allow fine contrast details to be differentiated at later timepoints [1104]. In another embodiment this background mapping may be carried out using the last frame in an acquisition when C-arm gantry or detector motion results in a different field of view and may preferably leverage previously illustrated embodiments to stitch together such background details with other frames across the new image region. Lumen filling [1105] is identified with knowledge of three-dimensional epicardial anatomy and regions are classified to identify regions of interest for microvessel function [1106]. The classified regions are tracked across multiple frames to identify changes in pixel densities representing contrast pooling/filling [1107] or dissipation [1108] over time to determine the residence time of contrast in the classified region and its associated intensity, made possible by prior knowledge of background structural or density properties. These metrics are used to calculate a virtual microvessel function score (vMF) [1110] which can be used with temporal contrast properties for subsequent steps including: determining left-right coronary dominance [1111]; mapping microvessel functional score to epicardual segments [1112]; and weighting boundary conditions for detailed simulation of biomechanical factors [1113].

Turning to FIG. 11B, the previous temporal contrast and virtual microvessel functional information may preferably be applied as weighted boundary conditions in detailed simulations [1114]. Illustrated is the method of determining standardised and easily comparable epicardial metrics between patients. Such a process overcomes the significant challenge of handling the impact of large variation in simple properties, such as blood pressure and blood velocity, and how these subsequently impact biomechanical based metrics preventing set ‘cutoff’ value(s) from providing the necessary prognostic value. The illustrated embodiment presents information on the morphological properties of scalar metrics, such as intraluminal helical flow in the current example [1115], across a range of augmented boundary conditions and in relation to vascular anatomy within a consistent domain ranging from 0 to 1 for absolute values or −1 to 1 for other properties. One skilled in the art will acknowledge the current approach is also applicable to assessing other vascular metrics. Here a cross section from intravascular imaging [1116] shows the lumen with cross-sections of an isosurface of counter rotating helical flow and its associated location in the three-dimensional vessel [1119]. One larger [1117] and one smaller [1118] cross section represents larger and smaller areas associated with a specified magnitude of the helical flow metric. In another region the two counter rotating regions show similar cross-sectional area [1120]. Taking the bounded ratio between the two rotating areas or preferably taking the ratio of the total isosurface cross-sectional area against the lumen area will result in bounded domains from −1 to 1 and 0 to 1, respectively, irrespective of the helical flow magnitude, blood velocity or other anatomical or physiological factors [1121]. Such approaches we name ratio of intraluminal flow to area and ratio of intraluminal flow imbalance. Here different values of the isosurface magnitude for helical flow are represented, where gradient variations [1122 and 1123] are also extracted as a single continuous variable over the length of the vasculature. Augmented boundary conditions [1124] such as to replicate functional cardiac output/loading can add multi-dimensional outputs [1125] still constrained within the same domain allowing a rapid assessment of virtual functional anatomy of the vasculature and its subsequent gradients [1126].

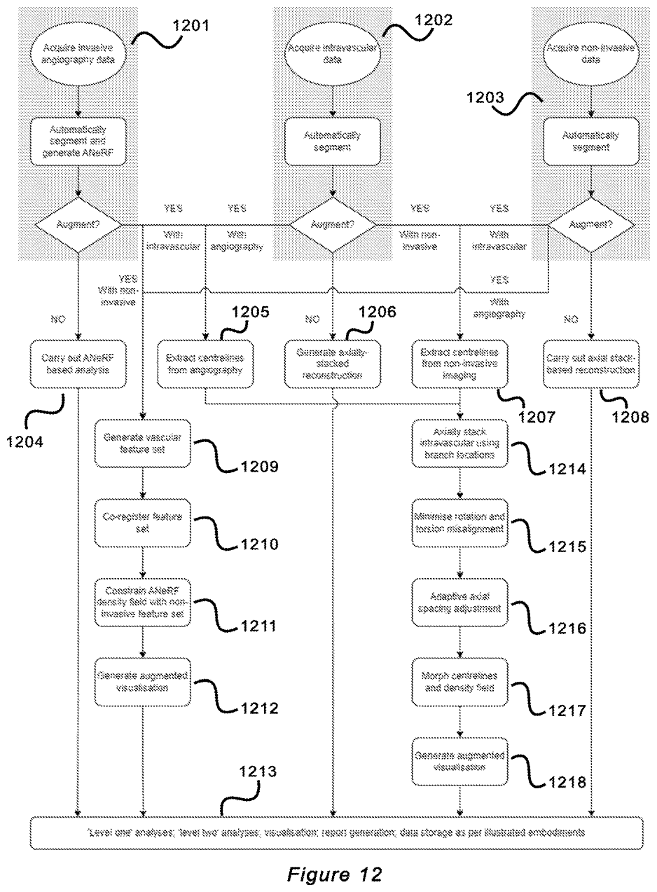

FIG. 12 illustrates a flowchart for the co-registration or augmentation of multiple imaging modalities into a single spatio-temporal model for both analytic and visualisation purposes. The flowchart may preferably take invasive angiography [1201], intravascular [1202] or non-invasive imaging modalities as inputs. Each individual modality undergoes its own pre-processing and segmentation processes separate from other processed before a manual decision is made to augment the data with another set of imaging data. In the case of invasive angiography, if no augmentation is to take place the standard ANeRF based processing is carried out [1204] before moving to subsequent analytic processes [1213] as outlined in the embodiments. If augmentation is chosen and non-invasive imaging such as computed tomography is available, both sets of processed imaging are passed to generate two vascular feature sets [1209] including tree-based anatomical structuring to co-register the two generated vascular models by minimising the distance between the two feature sets [1210]. As computed tomography is often performed before invasive angiography as a decision-making step, the ANeRF is then modified to leverage to co-registered feature set to constrain the three-dimensional density field to known vascular structures for the patient [1211], improving the speed and accuracy of the density map generation. The augmented visualisation is then produced [1212] and data proceeds to subsequent steps whereby the additional information, specifically plaque related composition and structure from non-invasive imaging is added to both ‘level one’ and ‘level two’ analyses. In another embodiment invasive angiography is chosen to be augmented by intravascular imaging by extracting vasculature centrelines using volume thinning operations [1205] and orientating the segmented intravascular stack along the vasculature using identified branch regions to first minimise a distance function [1214]. After distance minimisation, an angular rotation function is minimised that takes into account torsion along the intravascular catheter during acquisition [1215] followed by an adaptive axial spacing adjustment [1216] that allows axial spacing between intravascular frames to differ between vessel segments that are split by visible epicardial branches. A final angiographic branch morphing step [1217] deforms the branch centreline and density field to match the orientated intravascular data to improve branch region morphology before generating the visualisation [1218]. The multi-step orientation procedure was produced to rapidly improve processing speed over a single step that contains all the processes. The same procedure is carried out to augment non-invasive imaging with intravascular data [1207]. Both intravascular [1206] and non-invasive imaging [1208] carry out a similar axial stacking procedure to generate three-dimensional vascular models if no augmentation is selected.

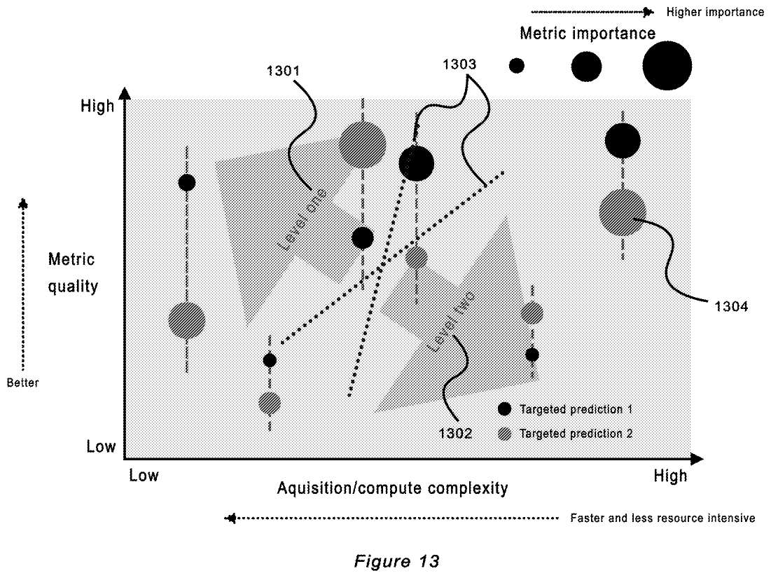

FIG. 13 illustrates the selection of data or features for the ‘level one’ and ‘level two’ analyses. The use of various metrics in the ‘level one’ and ‘level two’ analyses is calculated using a balance between acquisition or compute complexity (including but not limited to time or difficulty in acquiring data including human capital, compute time, computer hardware requirements and/or network bandwidth), metric quality (including but not limited to assessing overlap or entropy in input data, noise or missing values) and metric importance (including but not limited to node purity or GINI feature importance). ‘Level one’ analyses [1301] preferably target near real-time analytics (low compute complexity) while ‘level two’ analyses [1302] preferably target metrics with larger computation complexity but may also include detailed analyses of metrics include in the ‘level one’ domain. Metrics will present differing importance and quality for different targeted predictions [1304] leading to an adaptive cutoff region [1303] between ‘level one’ and ‘level two’ analytics depending on the targeted prediction or outcome and the acquisition/compute complexity including considering varying hardware availabilities.

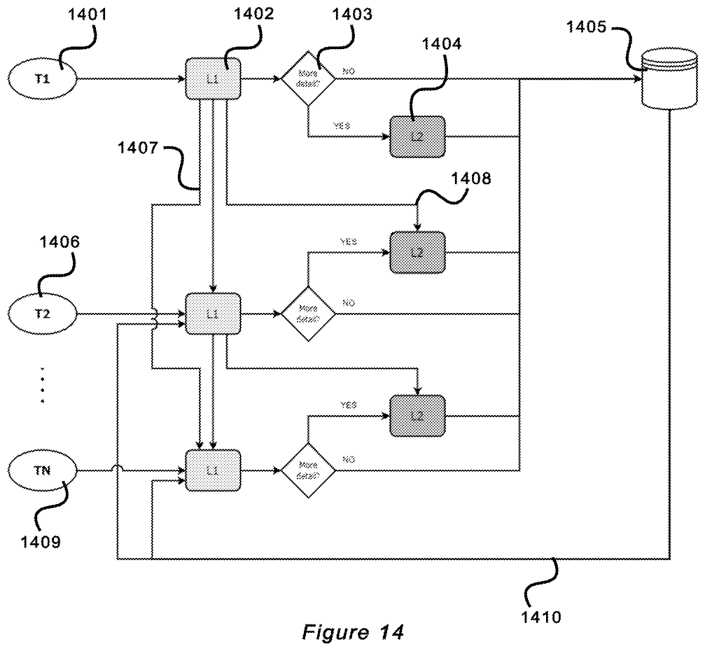

FIG. 14 is a schematic of logging multiple events or data inputs over time for a single patient within the proposed embodiments. At the first admission timepoint [1401] patient data is processed via previously discussed embodiments and a ‘level one’ (L1) analysis is carried out [1402]. Upon preferably automated selection but also user/manual selection a decision to proceed to a ‘level two’ analysis (L2) is made [1403] and a detailed L2 analysis is carried out [1404]. Both analysis L1 and L2 are transferred over a network to a storage pool [1405] either located locally or in a cloud environment. Upon a second admission [1406] for analysis at either a different timepoint or for a different procedure and/or image analysis acquired data is passed for subsequent L1 analysis. Here it should be noted that data from previous L1 analysis irrespective of which imaging modality led to the previous analysis is incorporated into the subsequent analytics [1407] to improve personalisation and predictive capability. It should also be noted that the continuous learning of the various levels of analysis provides an updated L1 analytic process via retrained models and/or weights by incorporating all levels of data that precedes the current timepoint [1410] and was available to use from the electronic database [1405] or embodied networks allowing recalibration of predictions and/or analytics with current patient characteristics. The same feed-forward process passes both the current and previous L1 analytic data to subsequently chosen L2 analyses [1408]. The process is repeatable with subsequent time points where the patient is analysed with one or several of the supported imaging systems or acquired data [1409] and benefits from continuous system learning based on previous admissions.

FIG. 15 is a flow diagram of the machine learning decision making approach incorporating the simulated biomechanical based metrics, machine learning based analytics and patient data to capture nonlinear interactions and features of a patient's complex condition(s). A plurality of metrics including continuum mechanics inspired metrics or those illustrated in the enclosed embodiments including transient variability over the cardiac cycle may preferably be taken as inputs to the machine learning decision making process. In one embodiment these metrics may preferably include information on the vascular structure [1501]. In another embodiment these metrics may preferably include continuum mechanics inspired metrics at or within the vascular wall including throughout vessel structural layers and plaque components [1502]. In another embodiment these metrics may preferably include haemodynamic properties throughout the vascular volume (i.e. not wall based quantities) [1503]. In yet another embodiment these metrics may include one or several of the features and their derivations illustrated through the enclosed embodiments. The metrics may then preferably be passed to a multi-level, multi-variable feature binning process [1504] with equal feature discretisation across bins. The number of bins and features throughout bins may preferably vary and be subject to automatic adjustment to optimise or maximise inter-and intra-bin variation. Detailed simulation metrics may often produce highly skewed or unbalanced distributions with small features (either small in terms of time, space or feature magnitude) often containing highly relevant pieces of information that may be missed or overlooked in many scenarios. Hence, in another embodiment the metrics probability distributions or other statistical distribution(s) describing the spread of each metric may be used to discretise inputs into bins [1505]. This statistical distribution may include multi-level distribution of specific spatio-temporal regions to enhance important and/or nonlinear feature extraction [1506]. The multi-level, multi-variable binning may preferably include and be optimised using patient characteristics that may preferably be taken as measurements such as varying blood test results (e.g., troponin level, lipoprotein, and a plurality of other measures). Such characteristics may also include sex, age, weight, body-mass index, clinical presentation including but not limited to STEMI, NSTEMI, MINOCA and stable or unstable patient status, medication usage and a plurality of others. In one embodiment these characteristics may be used as weights or ‘levers’ to move bins for optimal capture of data from one or several metrics. Such movement may include metrics not being binned on one or several levels [1508]. In another embodiment these bin layers may be fixed based on set requirements from ‘level one’ or ‘level two’ analyses as illustrated in previous embodiments. In another embodiment these bins are used as input or hidden layers in a fully connected network such that binned distributions are available to some or all other bins within the feature set. In another embodiment metric inputs may skip a binned layer as deemed fit using the ‘lever’ action of various patient characteristics and may feed continuous spatio-temporal data into various layers of the fully-connected network [1507]. Various layers of the multi-level, multi-variable binning process may impose multi-variable weights to either entire bins or data captured within bins or each layer or connection of the fully connected network. Weights may also be applied to each metric before being input to the binning or fully connected network. In another embodiment the fully-connected network may automatically prune connections to optimise the propagation of features through the network. Pruning may preferably produce parallel pathways [1509] for feature propagation and enable multiple parallel endpoints [1510] to be acquired simultaneously including but not limited to the likelihood of an outcome, the location or statistical probability of a certain feature being present and the probability or predicted success rate of one or several intervention or treatment pathways. In one embodiment these pathways may include all available weighted metrics. In another embodiment these pathways may include one or several metrics and may preferably differ between the parallel outputs being assessed [1511].

FIG. 16 is an example overview of the intravascular imaging visualisation and user interface. Turning to FIG. 16A the user interface contains a panel for visualising and interrogating data storage systems for patient data [1601] including accessing one or various imaging modality types from one or several time points or sites/clinics. The main fully interactive visualisation interface adaptively changes to suit the selected imaging with the presented embodiment an example of intravascular optical coherence tomography imaging. The user has the capacity to adjust through use of a mouse pointer or touch screen interface the automatically segmented or classified image structures. The interactive panel [1603] provides further functionality for the user to trim the imported image stack or select one or several machine learning approaches to apply to the image stack of which several have been defined in the previous embodiments. The longitudinal vessel map presented along the lower panel contains an interactive slider to drag through the image stack [1605] and presents information from each image of the entire pullback as a semi-three-dimensional visualisation that includes the cross-sectional area of the vessel and in one embodiment locations and volume of lipidic or calcified plaque [1606]. In another embodiment these features may be adapted by the user to define other plaque related measurements such as fibrous cap thickness along the longitudinal map. In another embodiment the longitudinal map also contains branch vessel locations [1607] and imaging catheter misalignment over the length of the vessel [1608] or may include one or several other features that can be graphed along the length of the vessel/image stack with various shape or colour features to identify specific metrics or details in either two or three-dimensions. Analysed data can be exported to electronic storage media or to third party applications. The user may also select to render the vessel structures in three-dimensions [1609].

Turning to FIG. 16B the user interface may present in three-dimensions the axial stack of the intravascular pullback which in the present embodiment visualised optical coherence tomography. The three-dimensional interactive visualisation may preferably take inputs from a mouse cursor or from a touch screen interface to view the vessel from any orientation including from within the vessel. In one embodiment the three-dimensional visualisation may show the lumen (blood component) while in another embodiment lipid and calcific components may also be shown [1611]. In another embodiment other structural features such as the layers of the vessel wall may be visualised and may be interrogated by the user. In another embodiment simulation data may be visualised as streamlines or pathlines of particle tracers representing blood flow through the vessel or as glyphs or manifolds for higher order tensor values throughout the blood and artery wall and plaque domains. An interactive three-dimensional slider [1610] may preferably move by the user's mouse pointer or touchscreen interactions and will visualise frame or slice based metrics in an adaptable popup visualisation that can be modified by the user to suit their preferences. In one embodiment this popup visualisation may preferably show the two-dimensional image frame [1613] with or without machine learning segmentations overlaid on the image or data relating to various features of the vessel [1614] and the relative metrics in the currently selected or interrogated frame such as fibrous cap thickness (FCT) overlying lipidic plaques or virtual percent atheroma volume (vPAV) calculated from previously illustrated embodiments. In another embodiment this popup visualisation may preferably show the calculated risk profile from ‘level one’ analyses such as the risk rating for various changes typically seen in coronary vessels and plaques and their associated statistical standard deviation or variance or confidence intervals [1615]. In another embodiment this visualisation may display demographic comparisons to rank patient-specific metrics or analytics or predictive results against a database of similar or different patient characteristics. In another embodiment a predictive model may be presented as a suggested treatment pathway. Such visualisations may preferably be customisable by the user who may select from individual metrics used in various levels of analytics from the embodiments or from overall risk scores that combine these metrics.

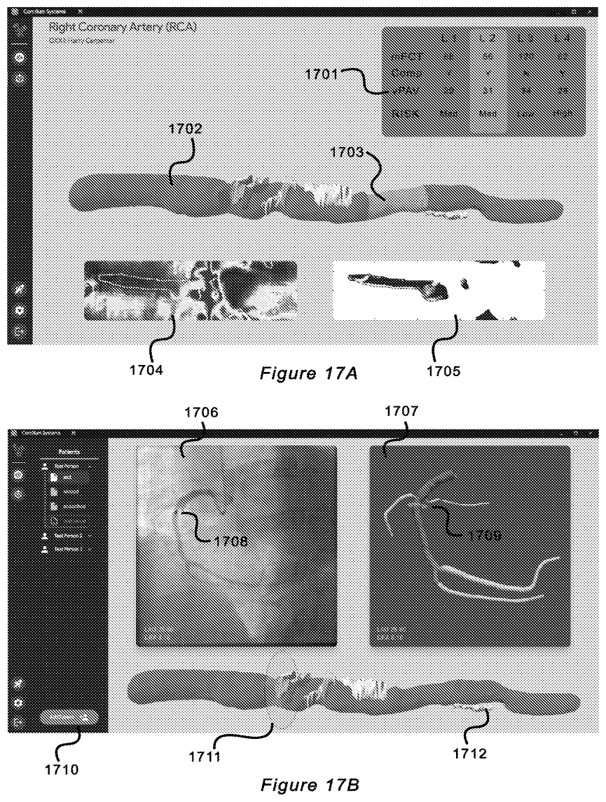

FIG. 17 is a further example of a simplified user interface with predictive and demographic comparisons. Turning to FIG. 17A data relating to various plaque features may preferably be displayed in tabular format [1701] and may change appearance or colour or contain other appreciable visual markers that change as the user interacts with different three-dimensional plaque features in the central visualisation [1702]. Predictive or analytic results may preferably highlight with colour or transparency or other appreciable techniques areas of the vasculature that are at risk of one or several outcomes [1703]. The user may interact with the highlighted region(s) with the mouse cursor or through touch screen interactivity including visualising two-dimensional views of the vessel wall which are ‘unwrapped’ from the vessel wall and may preferably present data on specific metrics or markers or predictive models as colour contours [1704]. In another embodiment the two-dimensional contours may preferably present data specific to plaques or data within the vessel or plaque structural features such as fibrous cap thickness [1705]. Turning to FIG. 17B, an interactive user interface is presented that augments intravascular imaging with invasive coronary angiography in real-time. The traditional two-dimensional angiographic frame(s) [1706] are visualised with C-arm orientation data and the subsequent three-dimensional visualisation of the vasculature is also shown [1707]. In one embodiment this three-dimensional visualisation is fully interactive for the user who can rotate and pan and zoom through the three-dimensional view with the associated c-arm specific view angle also displayed. In another embodiment both the two and three-dimensional views may be transient in nature and are able to show the function of the vessels over time. In yet another embodiment the three-dimensional view may be colour coded to visualise regions of interest such as the location of the intravascular imaging region as depicted. The three-dimensional axially-stacked pullback data from previous embodiments may also be visualised in three-dimensions [1712] with varying level of detail. In another embodiment this visualisation may include the two-dimensional ‘unwrapped’ visualisations from previous embodiments. In yet another embodiment the user may interact with either of the three main views and move a shape or colour-based marker [1708, 1709 and 1711] that registers the locations between various imaging types and visualises this in real-time. In another embodiment this co-registration may be automated and in real-time to view additional catheters being inserted through the vessel(s). In another embodiment several imaging modalities or acquisitions may be manually selected to add or augment with the visualisation from available patient data [1710].

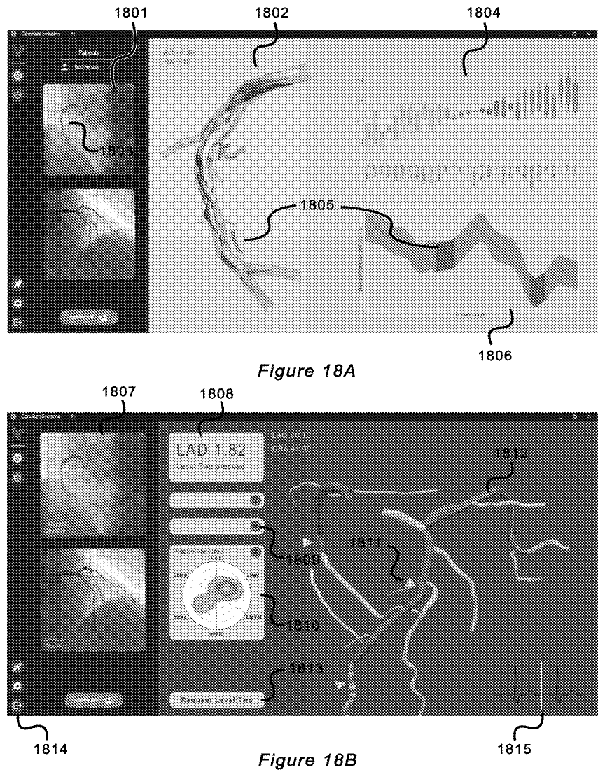

FIG. 18 is exemplary of the augmented user interface containing data from multiple modalities, analytics and/or predictive results in a single interface. Turning to FIG. 18A, multiple angiographic views are presented [1801] for interactive selection of one or several vessels or vessel sections to interrogate available metrics. A three-dimensional representation of the selected vessel and various analytic or simulation metrics can be interacted with by a user [1802] including rotation, zooming and panning, with the selected vessel or segment highlighted for reference on respective images [1803]. Various metrics from one or both ‘level one’ and ‘level two’ analyses may be available for viewing [1804]. In one embodiment the metrics may be interactive allowing cursor or touch interaction to view key data points or selection for visualisation on the interactive three-dimensional view of the vessel. In another embodiment these metrics may be interrogated in further detail such as over the length of a vessel or segment of interest [1806]. The illustrated ratio of haemodynamic instability automatically highlights regions of interest or regions with predictive significance and may preferably highlight these on the interactive three-dimensional view [1805]. One skilled in the art may appreciate the benefit from being able to automatically interrogate any individual metric as well as a combination of metrics that lead to predictive analytics such as the embodied biomechanical stress profiling index from ‘level one’ and ‘level two’ analyses.

Turning to FIG. 18B yet another interactive visualisation is presented that augments data from multiple imaging modalities and both ‘level one’ and ‘level two’ analyses. Multiple invasive angiographic views are presented including one each from the left and right coronary trees [1807]. In another embodiment several angiographic views may be presented in this window. It should be appreciated that other imaging modalities such as non-invasive computed tomography may also be visualised in this section in various forms such as with axial, sagittal and coronal plane views rather than in C-arm specific coordinates. Both the left and right coronary trees may preferably be visualised from as little as two angiographic views (one each for left and right) and are fully interactive through various user inputs such as via a mouse pointer or touch screen interaction allowing zooming, rotating, panning and other three-dimensional interactive processes. The vasculature may be colour coded to present additional information to the user such as predictive results or regions of intravascular imaging pullbacks [1812]. In another embodiment the three-dimensional visualisation may include shape-based markers or other visually appreciable methods to highlight specific regions or data point designed as important for the user [1811]. Such markers may be interactive and may display additional information such as predictive graphs or datapoints [1810]. Such additional data may preferably be dynamically and automatically displayed in such a way as to first show the most important data to the user by using the most critical pieces of information designated or extracted from the decision-making process in previous embodiments rather that a static data point. In one embodiment this may include the key outcome or biomechanical stress profiling index [1808] or in another embodiment it may include automatically opening additional drop-down menus [1809] or similar methods to display an otherwise hidden outcome [1810]. Other data points or metrics are also presented and can be accessed from various menus [1809] including allowing the user to specify a specific data layout tailored to their needs that may be saved as a user preference. The input data may be gated to the electrophysiology of the heart [1815] to identify phases over the cardiac cycle and allow transient display of the vasculature rather than only a static model from one timepoint which may be further interactive for the user to identify changes throughout the cardiac cycle. Such capability allows real-time visualisation of additional wires such as those for intravascular imaging or for stent insertion to be visualised as they are inserted into the vasculature. Such visualisation may preferably allow interaction by the user including rotating views to improve three-dimensional visualisation of the inserted device and subsequent co-registration and visualisation on the two-dimensional angiographic images as presented in FIG. 18A.

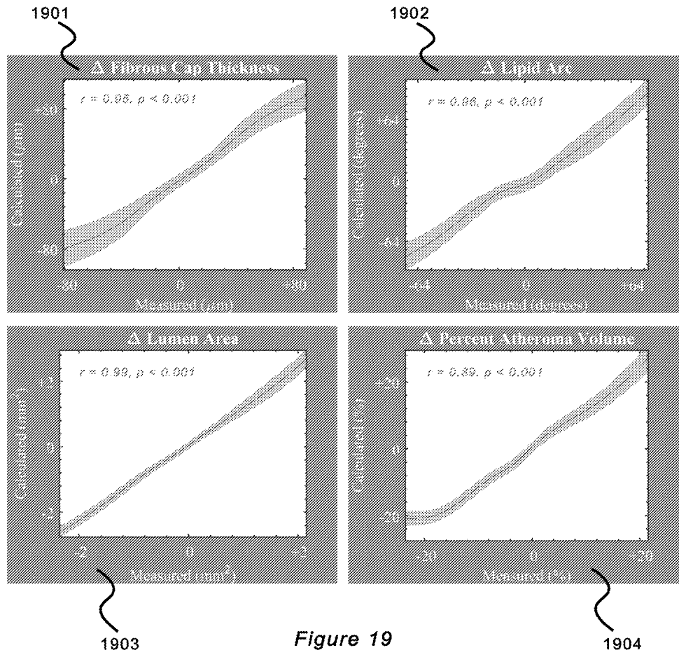

FIG. 19 illustrates examples of indicative performance of the enclosed embodiments to identify how plaques will change over time. Results demonstrate good correlation between estimated/calculated changes from the enclosed embodiments and measured changes from patients in an important but non-exhaustive list of plaque features. Images are visualised as locally weighted logistic regression fits with 95% confidence intervals and the strength of Pearson's r correlation for fibrous cap thickness [1901], lipid arc [1902], lumen area [1903] and virtual percent atheroma volume as determined through the exemplified embodiments.

It should be appreciated by those skilled in the art that previous methodologies could only identify physiologically relevant plaques with relatively weak discriminative ability. They could not predict the degree of change in the vasculature, which the illustrated and exemplified embodiments are able to achieve. They could also not distinguish between different types of changes (e.g., between a thinning fibrous cap of a plaque and a narrowing lumen) which the illustrated and exemplified embodiments are able to achieve.

There are several differences with the method of the present invention and the systems disclosed in the prior art. In one embodiment the systems and methods of the present invention uses invasive coronary angiograms (also known as contrast angiogram or biplane angiogram) to produce a predictive model including the recommendation of treatment or examination pathways.

In another embodiment of the systems and methods of the present invention intravascular imaging (here presented using optical coherence tomography) is used to develop a predictive model to recommend treatment or examination pathways.

In another embodiment of the systems and methods of the present invention non-invasive computed tomography is used to develop a predictive model to recommend treatment or examination pathways.

The reader should however appreciate that the steps of the present systems and methods could utilise further derivations of the mentioned imaging modalities without varying from the scope of the present invention and that the present systems and methods augment these imaging modalities into a single system rather than fragmented analytics.

The method of the present invention may also produce two levels of analysis; a real-time ‘level one’ and if identified as required by the ‘level one’ analysis a more computationally demanding ‘level two’ analysis to produce predictive models which differs from all previous approaches which take a set input to produce a single static output.

The method of the present invention may take inputs for a patient from multiple time points or previous examinations or differing imaging modalities to improve the analytics and further personalise the predictive model which other systems and methods are not able to accomplish due to their static nature.

The method of the present invention may use adaptive spatio-temporal machine learning segmentation model(s) and a customisable physics informed neural network within a single process to automatically identify vascular components and reconstruct regions with significant attenuation artefacts that were not previously possible.

The method of the present invention may produce a three-dimensional density map of the vasculature from as little as a single invasive angiographic frame via an angiographic neural radiance field (ANeRF) which differs significantly from both previous approaches to segment angiographic images and traditional neural fields, amplifying available information to the clinician while reducing radiation exposure to the patient.

The method of the present invention may further leverage the ANeRF to produce ventricular estimates including virtual ejection fraction (vEF) from angiography either with or without ventriculography further reducing radiation exposure and treatment time for the patient.

The method of the present invention may further use ANeRF to produce a transient and differentiable vascular density map over one or multiple cardiac cycles and assess physiological function from angiography images such as via defining a vessel specific virtual microvessel function (vMF) score or virtual vessel strain (vVS) which previously required an additional invasive wire to be inserted into the patient.

The method of present invention uses existing measured metrics and newly identified metrics to assess multidirectional stress. The method of the present invention utilises these metrics together with patient factors to generate a multi-dimensional risk score or index for identifying multiple probabilistic outcomes.

The method of present invention utilises a measurement set determined by combining an adaptively pruned or weighted set of the computed metrics adjusted by patient factors and vasculature characteristics to compute this risk score.

The method of the present invention provides an augmented visual display that may preferably combine and display visuals of the predictive results and imaging from one or all available imaging systems to amplify available information in the clinic.

The following provides further clarity on the terms relating to the continuum mechanics and vasculature metrics calculated and used throughout the description and claims. Further patient-specific data such as age, sex and clinical presentation among others are also accounted for in the predictive models.

The metrics that are marked ‘**’, denote metrics developed by the inventor in a novel embodiment of the present invention and that have to their knowledge not been previously used or calculated through such methods. Such metrics may have been calculated or acquired by previously existing invasive or alternative methods whereas the current embodiment presents a novel non-invasive approach(s) to determine or quantify these metrics. The other metrics are generally considered common knowledge in the fields of engineering and/or cardiology.

### Vessel Morphological Characteristics:

These metrics are automatically extracted from various imaging modalities, with highlighted metrics ** able to be extracted from intravascular optical coherence tomography without further imaging systems.
If angiography or computer tomography is available:
   Vessel volume, torsion, curvature, stenosis percentage, lumen area, lesion diffusivity, lesion length, branch angulation and ostium position.
If intravascular imaging is available, the previous characteristics may be supplemented with:
   Plaque and vessel morphology, including but not limited to: fibrous, lipidic, lipid rich, lipid arc, lipid volume, calcified, calcium volume, complex plaque, fibrous cap thickness, fibrous cap morphology, eccentricity, macrophage index, micro-vessels, cholesterol crystals, thrombus, intimal thickening, bifurcation morphology, lumen area and the nonlinear material properties of different components.
   Inner and Outer elastic membrane volume **: The cross-sectional area of both the inner and outer elastic membranes which not visible in traditional intravascular imaging due to light attenuation.
   Virtual percent atheroma volume (vPAV) **: An extension of the previous metric to provide a percentage ratio of the outer elastic membrane to lumen area used to identify plaque burden.
   Lipid volume **: The cross-sectional area or volume of lipid, not previously available in intravascular optical coherence tomography due to light attenuation but made possible with the illustrated embodiments.
If intravascular imaging is not available, the ‘level one’ and ‘level two’ analyses provide estimates of the previous characteristics using probabilistic methods.

### Functional Based Metrics:

These metrics are automatically extracted from invasive angiogram images **, removing the need to insert an additional wire in the patient.
Virtual microvessel function (vMF) **: A measure of the resistance to blood flow from the microvessels that stem from epicardial coronary vessels assessed from angiography images and the subsequent differentiable density map from the ANeRF.
   Contrast agent flow velocity and perfusion time: Calculation of blood flow velocity using contrast movement and the three-dimensional density map features including contrast dissipation rate.
   Thrombolysis in myocardial infarction (TIMI) flow grade: A risk score for future adverse cardiac events for people with unstable angina or NSTEMI presentation.
   TIMI myocardial perfusion (TMP): A flow grading system for perfusion in the myocardium.
   Blush residence time and intensity: Quantitative measures of the time and intensity of ‘blush’ presence on coronary angiograms and the association to contrast velocity and dissipation, assessed using features of the transient three-dimensional density map.
   Contrast pooling time: A quantitative measurement of contrast pooling time and severity in epicardial vessels to denote regions of slow or disturbed flow or to highlight regions of significant foreshortening.
Virtual vessel strain (vVS) **: A measure of the three-dimensional displacement of the epicardial vessels over one or several cardiac cycles by leveraging the differentiable and transient vascular density map.
Virtual ejection fraction (vEF) **: A quantitative measure of the output from the left ventricle calculated using the transient three-dimensional features of the vascular density map and optionally augmented with ventriculography.
Virtual pulse wave velocity (vPWV) **: A virtual calculation of vessel stiffness using transient features of the vascular density map and knowledge on blood properties and its physics.
Virtual arterial distensibility **: An extension of the vPWV to determine how vessels change over the cardiac cycle (i.e. how the contract and expand with cardiac function).
Virtual augmentation pressure **: A further expansion on vPWV, arterial distensibility and physics-based metrics to measure pressure wave reflection throughout vessels.

### Continuum Mechanics Based Metrics:

### Haemodynamics (Fluid Mechanics-based Metrics):

Wall shear stress: Frictional force between the wall of the vessel and blood flow, denoted as a vector and often presented as its magnitude along, calculated using the gradient of the velocity field and fluid strain rate, and with further derivations including: Wall shear stress gradient, time average wall shear stress, oscillatory shear index, relative residence time, transverse wall shear stress, cross flow index, axial wall shear stress, secondary wall shear stress, wall shear stress divergence, wall shear stress exposure time, critical point location and residence time, wall shear stress variation, and their subsequent normalised or transient variations over one or several cardiac cycles.
Helical flow: Helical flow is the ‘corkscrew like’ behaviour of blood flow through an artery. There are four different commonly used measures to quantify it, namely: H1, H2, H3 and H4.
Pressure: Used to assess the significance of stenosis (i.e. narrowing's). Currently measured using fractional flow reserve [FFR], requiring a pressure wire to be inserted in the artery, or quantitative flow ratio [QFR] which is non-invasively calculated using only angiogram images, both commercially available processes).
Blood velocity profile (magnitude and directions).
Ratio of intraluminal flow to lumen area **: A ratio of the effective cross-sectional area of the absolute intraluminal flow characteristics (assessed using the isosurface of any intraluminal flow measure such as velocity, helical based quantities or lagrangian coherent structures), over the cross-sectional area of the artery lumen (fluid component). The resulting metric is a geometric representation of a flow metric that is constrained everywhere to between 0 and 1, making comparison between patients more meaningful.
Ratio of intraluminal flow instability **: An extension of the previous metric as a ratio of the effective cross-sectional area of positive and negative intraluminal flow characteristics, resulting in a geometric interpretation of flow imbalance that is constrained to between −1 and 1 everywhere, making comparison between patients more meaningful.
Turbulent kinetic energy and its dissipation rate: Describes the mean kinetic energy per unit mass in turbulent blood flow.

### Structural Mechanics:

Cauchy stress tensor: The nine-parameter tensor which completely describes the stress state of a volume in a deformed body and its derivations including: Principal stress magnitude, principal stress gradient, axial principal stress magnitude and normalised misalignment (from the axial vector), secondary principal stress magnitude and normalised misalignment (from the secondary vector), radial principal stress magnitude and normalised misalignment (from the radial vector), tensor divergence, and their subsequent normalised or transient variations over one or several cardiac cycles.
Ratio of intrastructural stress to external elastic lamina area **: A ratio of the effective cross-sectional area of the absolute stress flow characteristics (assessed using the isosurface of any stress metric or invariant manifolds of the stress tensor), over the cross-sectional area of the external elastic lamina. The resulting metric is a geometric representation of structural stress that is constrained everywhere to between 0 and 1, making comparison between patients more meaningful.
Ratio of intrastructural stress instability **: An extension of the previous metric as a ratio of the effective cross-sectional area of positive and negative intrastructural stress characteristics, resulting in a geometric interpretation of stress flow and stress imbalance that is constrained to between −1 and 1 everywhere, making comparison between patients more meaningful.

The skilled addressee will now appreciate the advantages of the illustrated invention over the prior art. In one form the illustrated invention provides a method and an algorithm that assists a clinician in decision making and determining patient treatments, by providing a predictive model which provides a personalised biomechanical stress profiling index for the patient.

Currently, there is a missed opportunity to analyse and predict which disease will progress and cause recurrent admissions. The technology that has been developed overcomes several crucial limitations of imaging systems and may play a role in assisting doctors predict which disease will progress, allowing them to personalise treatment, keeping patients healthier and out of hospital, thereby reducing the cost burden to the community.

Various features of the invention have been particularly shown and described in connection with the exemplified embodiments of the invention, however it must be understood that these particular arrangements merely illustrate the invention and it is not limited thereto. Accordingly, the invention can include various modifications, which fall within the spirit and scope of the invention. For the purpose of the specification the word “comprise”, “comprises” or “comprising” means “including but not limited to”.

1. A computer implemented method of producing an advanced visualisation and predictive model to provide a personalised biomechanical stress profiling index for a patient including the steps of:
a. acquiring images, data and characteristics relating to the patient;
i. acquiring imaging information from one or more invasive catheter-based imaging systems; and/or
ii. acquiring imaging and gantry orientation and gating information from one or more planes in invasive coronary angiography and/or ventriculography; and/or
iii. acquiring imaging information from non-invasive computed tomography imaging; and/or
iv. acquiring continuous measurements including heartrate, blood pressure and electrocardiogram patient;
b. constructing a vasculature model of at least some of the patient's arteries;
c. extracting or calculating physiological information from acquired images, data and characteristics relating to the patient;
d. undertaking an initial ‘level one’ artificial intelligence and/or biomechanical assessment optimised for real-time analytics from local and/or cloud computer hardware and the available data;
e. using the ‘level one’ results to determine a theoretical optimal treatment or examination pathway or to assess the need for a ‘level two’ analysis;
f. generating an augmented visual display and/or report of the ‘level one’ results to assist a clinician in decision making;
g. undertaking a ‘level two’ artificial intelligence and/or biomechanical simulation tailored to support and enhance the ‘level one’ analysis for providing a comprehensive patient assessment using several metrics;
h. using the metrics, acquired imaging, data and characteristics relating to the patient to produce a continuous, multi-dimensional biomechanical stress profiling index of one or several plaques and/or arteries.
i. retraining and updating the ‘level one’ analyses with results from biomechanical stress profiling index;
j. utilising the biomechanical stress profiling index to highlight regions of vulnerable plaque, plaque composition, risk of future growth and destabilisation, and/or vessel changes over time; and
k. generating an augmented visual display and/or report of the index including one or several imaging modalities to assist the clinician in decision making and determining patient treatments.

2. The computer implemented method in accordance with claim 1, wherein the processing and extraction of key features from intravascular imaging being automated and further including the steps of:
a. Acquiring an intravascular imaging pullback/image stack and associated data at the time of acquisition including blood pressure, heart rate and imaging system physics based partial differential equations;
b. Pre-processing the image stack to remove unwanted regions and preferably prefilter noise or artefacts;
c. Passing the pre-processed imaging stack and acquired data/physics knowledge to general computing hardware selected from a group including a suitable graphical processing unit;
d. Segmenting the lumen using a spatio-temporal U-Net machine learning architecture that that leverages long-short term memory (LSTM) and attention mechanisms for robustness and generalisation strength in sparse and noisy real-world data;
i. machine learning model also applies dynamic vertical layering to modify the model layers during processing to improve segmentation; or
ii. adaptive block contraction is applied to further reshape encoder or decoder architecture during processing to improve segmentation;
e. Masking the preprocessed image stack with the resulting lumen segmentation map;
f. Feeding the masked and preprocessed image stack to a three-dimensional U-Net based machine learning architecture to segment the medial layer of the vessel wall;
g. Passing the preprocessed image stack, lumen segmentation map and medial layer segmentation map to a pre-processing module of a modified deep physics informed neural network architecture;
h. Processing the three-dimensional pixel location, pixel multilayer colour data and multiple segmentation maps through a stage one multilayer perceptron's;
i. Extracting global properties from the multilayer perceptron's and acquiring further data from the pre-processing module including a smoothed three-dimensional vessel centerline;
j. Passing local spatial information to a stage two multilayer perceptron's which have access to partial differential equations governing tissue continuity, nonlinear tissue properties, imaging physics-based properties and pressure distributions;
k. Imposing boundary or initial conditions on the network to aid convergence using segmentation masks from step ‘d.’ and step ‘h.’;
I. Minimising the network loss function to extract segmented tissue maps including in areas with significant imaging attenuation artifacts and information on tissue properties; and
m. Performing the above steps within a single operation on a graphical processing unit.

3. The computer implemented method in accordance with claim 1, wherein a three-dimensional density map or anatomical model of the vasculature from invasive coronary angiography is generated via an angiographic neural radiance field (ANeRF) to thereby minimise patient radiation exposure while amplifying available information to the clinician, and the method further including the steps of:
a. Acquiring at least one invasive angiographic view of the vasculature containing one or several images over the cardiac cycle;
b. Extracting C-arm orientation metadata from the acquired image data including primary and secondary angles, detector properties, x-ray properties and source location with respect to the patient/gantry icocenter and the detector plane;
c. Preprocessing the angiographic image or image stack with a machine learning model or numerical method to identify vascular structures;
d. Developing a multigrid or sub-pixel representation of the vascular structures identified in step ‘c.’ to improve render resolution of density map or anatomical model;
i. if multiple acquisitions from varying primary or secondary angles are present then steps ‘a.’ through to step ‘d.’ will be carried out on each acquisition; or
ii. if multiple views are aligned using their C-arm coordinate metadata extracted in step ‘b.’; or
iii. these views are aligned using an energy minimisation algorithm to overcome acquisition setting errors, c-arm gantry motion, patient motion including from breathing/heart movement and from table or detector panning;
e. Providing the multiscale representation of the angiographic image(s), binary masks and associated C-arm gantry orientation (after alignment with the energy minimisation algorithm) as inputs to the angiographic neural radiance field;
f. Rendering in three-dimensions the density field of the vasculature;
i. generating the three-dimensional density field with inclusion of three-dimensional vascular connectedness filters to enhance vascular structures and reduce noise; or
ii. processing the density field into voxelised or mesh-based visualisation techniques; and
g. Interactively visualising the three-dimensional anatomy for review by the clinician.

4. The computer implemented method in accordance with claim 3, for acquiring transient information from invasive coronary angiography imaging to determine virtual vessel, microvessel and ventricular function without the need for further tests or invasive wires, further including the steps of:
a. Developing the three-dimensional density field of the vasculature in accordance with claim 3;
b. Identifying background features across angiographic frames including ribs or spinal bones;
c. Applying rigid body transformations to co-register background features across image frames to account for C-arm gantry or patient motion;
i. co-registration produces an augmented set of images representing a two-dimensional space larger than any individual image frame; or
ii. the co-registration produces a variable set of C-arm gantry orientations to account for motion artefacts across several image frames;
d. Mapping forward and backward facing images from one or several angiographic frames to the static three-dimensional density field;
i. the co-registered image stack is used to generate a unique three-dimensional density field for each set of frames over time; or
ii. the static density field is encoded with continuity constraints and deformed over time to mimic the two-dimensional co-registered image stack;
e. Fitting a predefined myocardial map to the three-dimensional density field;
f. Deforming the fitted myocardial map over one or several cardiac cycles to estimate ventricular function selected from a group including ejection fraction;
i. a ventriculogram is available and used to optimise the predefined myocardial map or ventricular estimates;
1. Reprocessing the density field to extract volumetric changes in the density of vascular structures over time; or
ii. the angiographic neural radiance field (ANeRF) is modified with an additional multilayer perceptron and Navier-Stokes and continuity-based loss function(s) to encode blood dynamics to the vascular density field;
1. Calculating the dissipation or change in density of the vascular density field;
g. Mapping the dissipation or density changes to specific vessels or vessel segments or myocardial segments; or
h. nonvascular regions are interrogated for changes in density in two or three-dimensions; and
i. the identified dissipation or density changes are graded and mapped to vascular structures or myocardial segments as areas of ‘blush’ or microvessel dysfunction.

5. The computer implemented method in accordance with claim 1, providing novel intraluminal or intrastructural biomechanical based metrics that are tailored to specific patients and can be generalised and compared directly between various patients, further including the steps of:
a. Generating an augmented set of boundary conditions based on patient characteristics;
b. Carrying out a biomechanical simulation or machine learning implemented method to determine a continuum mechanics based metrics using an augmented boundary conditions;
c. Calculating isosurfaces of normalised metrics of interest which includes traditional or novel metrics from several equally spaced units within the domain −1 to 1 or 0 to 1;
i. Taking one or several plane-based slices of one or all isosurfaces from step ‘c.’ and determining the area contained within each plane based isosurface slice; or
ii. Taking one or several plane-based slices of one or all isosurfaces from step ‘c.’ and determining the area contained within the positive and negative isosurface plane based regions;
d. Determining the cross-sectional area of the vessel at one or several planes used in steps ‘c.i.’ and ‘c. ii.’;
i. Calculating the ratio of isosurface plane/slice based area to the lumen area or the ratio of area of the positive isosurface slice to the area of the negative isosurface slice from one or several domain units; or
ii. calculating the augmentation variability of the ratio of isosurface and/or lumen plane area across one or several domain units across the range of augmented boundary conditions imposed from step ‘a.’; and
e. Generating a visual display or graph or report of an augmented intraluminal biomechanical based metrics.

6. The computer implemented method in accordance with claim 1 for selecting, distributing and using available data to predict or identify outcomes or features in a patient's vasculature, further including the steps of:
a. Acquiring various input metrics identified in any one of claims 1 to 6;
b. Determining the statistical or probabilistic spatio-temporal distributions of continuous metrics;
c. Multi-level discretisation of the statistical or probabilistic spatio-temporal distributions to highlight or improve weighting on important locations or results;
d. Binning discretised or whole metrics in a multi-level, multi-variable feature binning process;
e. Weighting or shifting bins using patient characteristics for optimal capture of data from one or several metrics;
f. Implementing the bins as inputs or hidden layers in a fully connected network to capture nonlinear features and interactions;
g. Automatically prune connections to optimise the propagation of features through the network in parallel or in serial processes; and
h. Provide the likelihood of an outcome, the location or statistical probability of a certain feature being present or the probability or predicted success rate of one or several intervention(s) or treatment pathway(s) for multiple parallel endpoints.

7. The computer implemented method in accordance with claim 1, wherein step ‘a.’ includes:
i. the acquiring imaging information from one or more invasive catheter-based imaging systems is selected from a group including coronary optical coherence tomography; and/or
ii. the acquiring continuous measurements including relevant data from wearable technologies and patient characteristics; and/or
iii. acquiring manual inputs from experienced technicians or clinicians.

8. The computer implemented method in accordance with claim 1, wherein step ‘b.’ further including the steps of:
i. pre-processing a two-dimensional intravascular imaging stack on a computer medium selected from a group including a central processing unit or graphical processing unit (CPU or GPU);
ii. scaling and axially stacking the pre-processed and segmented image data into slices in three-dimensions;
iii. communicating the pre-processed and segmented data from the local system over a secure network to a centralised cloud compute or container instance;
iv. segmenting the pre-processed image stack on a CPU or GPU using machine learning selected from a group including a temporal or three-dimensional neural network to identify vascular structure;
v. classifying and segmenting vascular scaffold(s), if present, in two-dimensional frames and generating a three-dimensional map of the scaffold on a GPU with a generative machine learning model and knowledge of the scaffold design pre-insertion;
vi. implementing a deep physics-informed neural network on a GPU with knowledge of tissue continuity, vascular structure, blood pressure and image properties to reconstruct medial and adventitial layers in attenuated regions;
vii. classifying and segmenting plaque components using a three-dimensional neural network machine learning algorithm;
viii. interpolating and voxelising the segmented data slices;
ix. in another form step ‘vii.’ includes feeding segmented slice data into a neural field to produce a differentiable density map in three-dimensions;
x. generating adaptable mesh from the voxelised/density structure suitable for three-dimensional user interaction and simulation processes; and
xi. communicating the processed steps over a secure network back to the local system.

9. The computer implemented method in accordance with claim 1, wherein step ‘b.’ further including the steps of:
i. Pre-processing one or more temporal angiogram and/or ventriculogram acquisitions or image sequences on a CPU or GPU;
ii. communicating the pre-processed and segmented data from the local system over a secure network to a centralised cloud compute or container instance;
iii. segmenting epicardial vascular structures using numerical and/or machine learning based algorithms;
iv. inputting the pre-processed image sequence(s) and segmented vascular structure(s) and gantry orientation(s) metadata into an angiographic neural radiance field (ANeRF);
v. generating on a GPU, using the angiographic neural radiance field, a three-dimensional density map of vascular and/or ventricular structures;
vi. creating an adaptable mesh from the three-dimensional density map suitable for three-dimensional user interaction and simulation processes; and
vii. communicating the processed steps over a secure network back to the local system.

10. The computer implemented method in accordance with claim 1, wherein step ‘b.’ further including the steps of:
i. pre-processing a stack or stacks of computed tomography images and/or axial, coronal and sagittal planes and associated metadata;
ii. communicating the pre-processed and segmented data from the local system over a secure network to a centralised cloud compute or container instance;
iii. segmenting vascular and ventricular structures using numerical and/or machine learning based algorithms;
iv. identifying and segmenting vascular scaffolds using numerical and/or machine learning based algorithms;
v. identifying and segmenting plaque components using numerical and/or machine learning based algorithms;
vi. interpolating and voxelising the segmented data stack;
vii. creating an adaptable mesh suitable for three-dimensional user interaction and simulation processes; and
viii. communicating the processed steps over a secure network back to the local system.

11. The computer implemented method in accordance with claim 1, wherein step ‘c.’ further including the steps of:
i. acquiring and processing a temporal range of images rather than a singular image frame;
ii. analysing acquired or processed temporal image data using probabilistic programming and/or machine learning based algorithms; and
iii. extracting relevant image features as a five-dimensional feature set.

12. The computer implemented method in accordance with claim 1, wherein step ‘c.’ further including the steps of:
i. acquiring and processing a temporal range of patient data or characteristics rather that static data points;
ii. analysing acquired or processed temporal data using probabilistic programming and/or machine learning based algorithms; and
iii. extracting relevant data features as a multi-dimensional feature set.

13. The computer implemented method in accordance with claim 1, wherein step ‘d.’ further including the steps of:
i. collating acquired or extracted data into a feature set or sets;
ii. generating an augmented set of boundary conditions to simulate patient cardiac or vascular load;
iii. analysing the feature set and augmented boundary conditions using physics informed machine learning models to acquire a lightweight subset of estimated biomechanical metrics in real-time;
iv. analysing the feature set(s) using computational statistics and generative machine learning models;
v. visualising the acquired feature set(s), computational statistical models and approach for each data point; and
vi. producing a report or dataset for storage in a local or cloud based electronic medium.

14. The computer implemented method in accordance with claim 1, wherein step ‘e.’ further including the steps of:
i. analysing the feature set(s) from step ‘d.’ of claim 1 using computational statistics, probabilistic programming and/or generative machine learning models;
ii. presenting the feature set(s) and the underlying computational model(s) to the user;
iii. taking manual user inputs from experienced clinicians/technicians including selecting or adding appropriate data and computational models suited to the patient;
iv. forecasting a generalised risk profile for the patient;
v. generating a probabilistic scenario for various treatment option(s) and presenting the scenario(s) in a graded fashion from strongest to weakest option;
vi. using the generalised risk profile and probabilistic scenario(s) to recommend or not recommend the use of a detailed ‘level two’ simulation; and
vii. producing a report or dataset for storage in a local or cloud based electronic medium.

15. The computer implemented method in accordance with claim 1, wherein step ‘f.’ further including the steps of:
i. accessing the report and/or dataset in preceding steps of claim 1 from the electronic medium;
ii. loading the user profile or taking manual inputs and formatting the visual display to suit their preset settings;
iii. populating the visual display with the report and/or dataset(s) from steps ‘a.’ to ‘e.’ of claim 1;
iv. automatically highlighting or presenting in a visually appreciable manner the statistically significant or important probabilistic data points;
v. augmenting the display with five-dimensional (three-dimensional space, time, and other metrics) data from one or more acquired datasets;
vi. using colour, shape markers or other visually appreciable methods to interactively highlight important regions throughout the vasculature to the user; and
vii. taking user interaction to alter or enhance the display including opening or closing additional data displays or adding/removing datapoints from the five-dimensional display.

16. The computer implemented method in accordance with claim 1, wherein step ‘g.’ further including the steps of:
i. taking a user command to proceed to a ‘level two’ simulation process;
ii. packaging all data from steps ‘a.’ to ‘f.’ of claim 1, and communicating the packaged data over a secure network to a centralised cloud compute or container instance;
iii. generating a coarse and a fine mesh of the vascular structure including the lumen, plaque components, vascular wall and epicardial structures;
iv. Defining patient-specific boundary conditions to the mesh structure including blood properties and profiles, displacement profiles and electrophysiological profiles; and
v. undertaking a simulation at one time-point and/or one heartbeat and or several heartbeats using acquired and calculated patient data, using continuum mechanics principles selected from a group including a fluid-structure interaction technique, a fluid-structure-electrophysical interaction technique, a computational fluid dynamics technique and a solid mechanics technique to determine engineering-based stress measures in the vasculature.

17. The computer implemented method in accordance with claim 16, wherein step ‘h.’ further including the steps of:
i. constructing a feature set from the ‘level two’ engineering-based stress measures;
ii. applying probabilistic programming and machine learning based decision approaches to the ‘level one’ and ‘level two’ feature sets;
iii. calculating using step ‘ii.’ a continuous and multi-dimensional biomechanical stress profiling index on the coarse mesh from step ‘iii.’ of step ‘g’ in accordance with claim 16;
iv. extracting from step ‘iii.’ using generative methods a feature set of likely outcomes on the patient, vessel, and plaque level(s) at varying time intervals;
v. adding the ‘level one’ and ‘level two’ feature set(s) to a secure cloud based electronic storage medium; and
vi. communicating the processed steps over a secure network back to the local system.

18. The computer implemented method in accordance with claim 1, wherein step ‘i.’ further including the steps of:
i. retrieving the ‘level one’ and ‘level two’ feature set(s) from the secure cloud based electronic storage medium;
ii. calculating via the centralised cloud compute or container instance the variance and/or error between the ‘level two’ and ‘level one’ feature set(s);
iii. taking manual inputs from experienced technicians if variance/error exceeds a set threshold;
iv. retrieving feature sets from the secure cloud based electronic storage medium for all relevant patients;
v. retraining the machine learning based approaches from steps ‘a.’, ‘b.’, ‘c.’, ‘d.’, of claim 1 and the ‘level one’ analysis with the retrieved data from steps ‘i.’ and ‘iv.’;
vi. retraining models from step ‘b.’ of claim 1 with a cross-imaging modality data augmentation approach;
vii. pushing the retrained hyperparameters and/or new machine learning models to the cloud-based machine learning operations (MLOps) pipeline; and
viii. communicating updated parameters via an electronic network to the local systems.

19. The computer implemented method in accordance with claim 1, wherein step ‘k.’ further including the steps of:
i. Taking manual inputs to adapt the visualisation to each users' preferences;
ii. Visualising the two-dimensional image(s) stacks from one or several imaging modalities;
iii. Visualizing the three-dimensional vasculature from one or several imaging modalities;
iv. Identifying with shape or colour or other visually appreciable markers regions of interest or data points for the user;
v. Taking manual user interactions with markers to display additional information;
vi. Automatically selecting and displaying the most important data to the user by using the most critical pieces of information designated or extracted from the decision-making process, rather than a static data point.
vii. Presenting data or metrics in both three-dimensions or modified two-dimensional visualisations; and
viii. Allowing interactive inputs to move or rotate or zoom two and three-dimensional visualisation of the vasculature in space and time.
